(19) United States
(12) Patent Application Publication
(10) Pub. No.: US 2015/0328454 A1
(43) Pub. Date: Nov. 19, 2015

(54) ELECTRICAL NEUROMODULATION STIMULATION SYSTEM AND METHOD FOR TREATING URINARY INCONTINENCE

(71) Applicant: BIO HEALTH FRONTIERS, INCORPORATED, LAS VEGAS, NV (US)

(72) Inventor: PAUL LAMBERT, EL DORADO HILLS, CA (US)

(21) Appl. No.: 14/712,442

(22) Filed: May 14, 2015

Related U.S. Application Data
(60) Provisional application No. 61/993,038, filed on May 14, 2014.

(51) Int. Cl. A61N 1/36; A61N 1/372; A61N 1/05

(52) U.S. Cl. CPC A61N 1/36007; A61N 1/0514; A61N 1/37247

(57) ABSTRACT

A system and method are provided for using neuromodulation techniques and intravesical electrical stimulation to treat Urinary Incontinence and related bladder-system conditions. The system uses an electrical stimulation module, stimulation electrodes and catheters, and/or a measurement and feedback system to determine an electrical stimulation therapy program as a function of a pre-programmed library and, optionally, measured and patient-provided response data. IVES and other electrical stimulation signals are generated and conveyed to the patient via catheter electrodes placed in and around the bladder system and related nerves, nodes and motor control points. The system employs a variety of safety mechanisms, including safety algorithms, a one-time use catheter connection, and catheter electrical-shock protection mechanisms.

### CROSS-REFERENCE TO RELATED APPLICATIONS

The present application claims priority from co-pending Provisional Patent Application No. 61/993,038, filed on May 14, 2014 and entitled Electrical Neuromodulation Stimulation System for Urinary Incontinence; that application being incorporated herein, by reference, in its entirety.

### BACKGROUND OF THE INVENTION

1. Field of the Invention

The invention relates to a system and method for treating urinary incontinence (“UI”) and related medical conditions and, more particularly, to a system and method of treating UI and related conditions using various therapies comprised of improved neuromodulation techniques and intravesical electrical stimulation (“IVES”).

Urinary incontinence (“UI”) refers to a person's lack of urinary system control, resulting in involuntary leakage of urine. The International Continence Society (“ICS”) defines “incontinence” as “an involuntary loss of urine that is a social or hygienic problem, and that is objectively demonstrable.” In 2000, according to the National Association for Continence (“NAFC”), UI has been diagnosed in more than 200 million people worldwide and more than 26 million Americans, with up to half having bothersome or severe symptoms. A related condition, overactive bladder (“OAB”), affects about twice as many American adults.

UI is more prevalent in women than in men. In the United States, about 75% to 80% of those suffering from UI are women.

A. The Urinary System:

The adult human urinary bladder holds 300 to 800 ml of urine, depending on gender and size. As the bladder fills, mechanoreceptors in the bladder wall sense the increased pressure of the liquid and send a signal to the brain, which ultimately induces the urge to urinate. A healthy individual can both feel this urge as well as consciously control the process of micturition. Through both sympathetic and somatic nervous system responses, the person's nervous system coordinates the control of the smooth bladder wall muscle (i.e., the detrusor muscle) and the urethra (i.e., the outlet that allows urine to pass to the external urethral meatus). FIG. 1A illustrates a simple reflex loop model of normal urinary inhibition, in which the nervous system inhibits or relaxes the detrusor muscle while exciting or constricting the urethra; this coordination is called “bladder-sphincter equilibrium.”

FIG. 2 provides a more detailed view of the process of volitional micturition (i.e., self-controlled urination). This complex process involves many parts of the anatomy that are coordinated under a person's conscious and unconscious control. The micturition reflex involves the higher cortex of the brain (i.e., the pons), the spinal cord, the anatomical components of the lower urinary tract (“LUT”), and the peripheral autonomic, somatic, and sensory afferent innervation of the LUT.

During the filling phase, the passive distension and stretching of the bladder activate sensory nerves (i.e., the mechanoreceptors) that lead from the bladder wall via pelvic nerves (comprised of myelinated A-delta afferent nerve fibers) and join with the spinal cord at the sacral level (notated as “(1)” in FIG. 1B). Corresponding stimulation of Beta-adrenergic receptors result in sympathetic efferent nerve signals that relax the detrusor muscle (“(2)”). Nerve signals also reach the pontine “micturition center” (“PMC,” also called “Barrington's Nucleus”), a collection of cells in the brainstem, which regulates the micturition reflex by regulating urine storage and release. In response, increased urethral sympathetic response excites the urethral motor nerves, and decreases parasympathetic response, which causes contraction of the pelvic floor and urethra (“(3)”).

In contrast, during the urination phase, volitionally triggering the micturition action coordinates a different nervous system response. Because the smooth muscle bundles in the detrusor are not normally well interconnected electrically, the detrusor will not normally contract without activation by the dense innervation of efferent nerves in the thoracic or lumbar spinal cord region. Volitionally triggering the micturition action coordinates the nerve responses generated by the distension of the bladder wall with the person's conscious control within the PMC to activate the efferent nerve signals. Hence, this innervation of efferent nerve signals induces synchronous contractions of the detrusor (causing a rise in intravesical pressure within the bladder) and inhibits the urethral sphincter (resulting in its relaxation), which together permit urine to flow through the urethra.

B. Types and Causes of UI:

i. Idiopathic UI:

Idiopathic UI is the lack of urine control in otherwise healthy patients. Idiopathic UI is generally classified as “stress incontinence,” “urge incontinence,” or “mixed incontinence” (which have elements of both stress and urge).

ii. “Stress UI”:

Stress UI involves the involuntary leakage of urine upon exertion. Such exertion may include coughing, sneezing, or exercising. Stress UI, a condition when urethral sphincter, can be caused by ineffective closing of the urethra due, for example, to pelvic floor muscular can no longer prevent leakage of urine. This can occur after childbirth or after pelvic surgery.

In Stress UI, leakage occurs because the abdominal pressure on the bladder is greater than the urethral pressure in the absence of detrusor contractions. Once this pressure (called the “leak point pressure”) is reached, the urethral sphincter can no longer prevent leakage of urine.

iii “Urge UI”:

Urge UI describes the presence of involuntary leakage of urine preceded by a sensation of bladder fullness and the need to void, and impending urinary loss. These sensations may cause “frequency” (i.e., voiding 8 or more times per 24 hours), and is associated with Urge UI as well as OAB. OAB frequently also causes “nocturia” (i.e., awakening two or more times per night to void). Urge UI can be categorized as including motor urgency, sensory urgency, and urethral instability.

“Motor Urgency” is OAB when confirmed by urodynamic testing. It includes idiopathic detrusor instability (“DI”), including DI by psychosomatic causes. Motor Urgency is also possibly associated with Bladder Outlet Obstruction (“BOO”) (e.g., due to prostatic obstruction or following surgery for stress incontinence). Motor Urgency may also be caused by neuropathic conditions (e.g., detrusor hyperreflexia).

“Sensory Urgency” induces the urgency response due to lower tract health conditions (e.g., stones and infections) or other idiopathic causes. Sensory Urgency may also be diagnosed in the absence of demonstrable DI in a patient.

“Urethral Instability” results from pathologic fluctuations in the urethral closure pressure at rest during the storage phase of micturition. Urethral instability may contribute to Stress UI or Urge UI.

Stress UI is more prevalent than Urge UI. Stress UI affects an estimated 50% to 75% of women in the U.S. while Urge UI affects about 25% women. Stress UI affects about 15% men in the US (mainly after prostate surgery or neurological injury) while Urge UI affects about 8%.

While the prevalence of Stress UI is higher, Urge UI is usually considered to be more bothersome.

iv. Detrusor Instability:

Bladder hyperactivity and spasmodic bladder (generally referred to as “unstable bladder”) is the uncontrolled contraction of the bladder wall (i.e., the detrusor muscle).

This condition can also be termed detrusor overactivity, detrusor hyperactivity, or “DI”. The bladder wall consists of smooth muscle bundles that are normally not well electrically interconnected. In the case of unstable bladder, these muscle bundles exhibit more prevalent electrical connectivity. This connectivity allows the spread of electrical activity and the possibility of uninhibited contractions within the bladder, which may lead to incontinence.

DI may also be associated with neurologic diseases, such as multiple sclerosis, Parkinson's disease, and “phasic hyperreflexia.” The latter involves contractions of the detrusor, which occur during the filling phase, and is often seen in younger women with Urge UI.

v. Functional Incontinence:

A category of nerve-mediated UI is “Functional Incontinence,” which refers to the urination urge at inconvenient times or inappropriate places with no obvious urinary system dysfunction. This may be caused by Alzheimer's Disease, mental deficit, or head injury. Similarly, “Reflex Incontinence” is the emptying of the bladder when a person's bladder contracts without the person being able to stop it, often without the ability to feel when the bladder is full. This may also be caused by a neurological condition, traumatic brain injury (“TBI”), or spinal cord injury.

vi Neurological UI:

In addition to neurological diseases causing DI, neurological UI can be due to birth defects (e.g., myelodysplasias, including spina bifida), spinal cord damage or TBI, or other neurological conditions that block pathways between the urinary system and the brain. This condition is sometimes referred-to as “neurogenic bladder.” Patients suffering from neurological UI cannot feel bladder fullness, and do not have conscious control over their detrusor muscle and/or urethra; such patients have the dangerous possibility of bladder distension if their bladder becomes overfilled.

Prolonged bladder distension can result in destruction of the mechanoreceptors (sensory nerves) in the bladder wall, with loss of bladder sensation. Children with myelodysplasia who have bladder decompensation from chronic over-distension have exhibited “lazy bladder syndrome”—a condition characterized by a large capacity bladder, and associated with a significant volume of residual urine after micturition.

vii. Irritable Bladder:

Apart from neurological UI, “irritable bladder” is a chronic condition due to interstitial cystitis. This can cause involuntary contraction of the detrusor, resulting in urge UI.

Various therapies are available to treat UI, ranging from behavioral treatments (such as physical therapy and specific exercises), pharmaceutical treatments, non-invasive treatments, minimally-invasive procedures, and surgical procedures. The efficacy of a particular treatment varies and is usually correlated to the degree of invasiveness of the treatment and its risk of complications.

C. Overview of the Current UI Therapies and Treatment Solutions:

A wide range of urinary incontinence therapies is available, although none has yet proven to be ideal. Despite the wide prevalence of UI and the many different therapeutic options available, most of the options are either relatively ineffective, have severe side-effects, or involve surgical risk.

Following are descriptions of the various types of treatments for Stress, Urge, and Mixed UI, and summaries of their effectiveness.

i. Home Therapies:

Patients may be first provided information regarding lifestyle modifications that may improve their UI, such as minimizing caffeine, alcohol and spicy foods, and quitting smoking.

Patients may also learn to perform to improve pelvic floor function, such as “Kegel” exercises. Biofeedback following treatment has been shown to increase the efficacy of such exercises.

Some UI patients may also try acupuncture to improve sensory and nervous activity in the bladder system.

Intravaginal (or vaginal) or anal electrical stimulation (“VES”) may be performed as a home therapy, but typically may be performed as an office procedure due to the number and intensive nature of treatment sessions needed. These techniques use surface electrodes, anal and vaginal plug electrodes, and dorsal penile nerve electrodes. While early clinical experimentation proved promising for controlling Urge UI, the intensive nature of multiple treatment sessions has proved less reliable at achieving and maintaining UI improvement. Biofeedback following treatment has been shown to enhance the rate of efficacy of VES.

ii. Pharmacologic Therapies:

Anticholinergic drugs block neurotransmitters in the peripheral and central nervous system. They inhibit parasympathetic nerve impulses by selectively blocking their communication to receptors in nerve cells and inhibit involuntary movements of urinary tract smooth muscles.

iii. Physician's Office Procedures:

A variety of office-based procedures are offered for patients with UI. They are described in the following paragraphs in increasing order of invasiveness.

Extracorporeal Magnetic Innervation which is intended to strengthen the pelvic floor without requiring the patient to make any effort, as by a sort of automatic Kegel exercise.

Vaginal cones are weighted devices that are inserted into the vagina and held in place consciously by the pelvic floor muscles of the patient.

Urethral inserts consist of a silicone tube with a mineral oil-filled sleeve and balloon. The device is inserted into the urethra and left in place until the next voiding; it is a single-use disposable device.

Percutaneous Tibial Nerve Stimulation (“PTNS”) is intended to treat women with OAB and associated symptoms of urinary urgency, frequency and Urge UI by stimulating the tibial nerve via insertion of a needle electrode.

Near-infrared laser therapy consists of shining low-power laser light on the pelvic floor (and/or vaginal vault).

Intravesical Electrical Stimulation (IVES) involves the use of a non-implantable urological catheter-like device with the ability to deliver electrical pulses to the inside of a patient's bladder via intravesical electrical stimulation pulses.

iv. Outpatient Procedures:

Radio frequency bladder neck suspension technology consists of an RF generator and bipolar applicator.

v. Implantable Devices:

Implantable stimulation devices exist that provide stimulation. A pulse generator is placed within the patient's body and subsequent adjustments of the stimulator impulse settings may be accomplished with the use of a remote electronic programming device.

vi. Surgical Procedures:

The most invasive form of urinary incontinence treatment is surgical, comprising various sling procedures being the main surgical option. These include the tension-free transvaginal (“TVT”) sling, the transobturator tape (“TOT”) sling, and the mini-sling procedures. These procedures require two abdominal incisions and one vaginal incision to maneuver a polypropylene mesh tape under the urethra to provide support that is not being provided by the pelvic floor.

The artificial urinary sphincter (in various forms) is one of the most invasive options, implanting a donut-shaped sac around the urethra and filled with saline or deflated to allow urine to pass.

In short, UI affects more than 200 million people worldwide. Stress UI, Urge UI and Mixed UI occur in a large percentage of adult women, although only about 10% are treated for their condition. UI therapies range from non-invasive (e.g., Kegels and biofeedback) to highly invasive (e.g., surgery), resulting in a range of efficacies and side-effects.

The micturition reflex involves both nerves and muscles in a complex physiological balance, involving the lower urinary tract, spinal cord, and brain. The science of UI has developed rapidly over the past few decades, resulting in a detailed understanding of the neural pathways and the central and peripheral neurotransmitters involved in urine storage and bladder emptying.

Overall, it can be seen that increasing invasiveness may come with a corresponding increase in effectiveness, but with potentially higher complications. FIG. 1C shows a table summarizing most of the common UI therapies, an approximate degree of invasiveness, and range of efficacy and complications commonly reported in the literature.

2. Description of the Related Art

A. Introduction:

Concepts of using various apparatus and methods for stimulating certain nerves and muscles to improve micturition performance have been previously suggested by others. Both pharmacologic and electrical neuromodulation approaches have been focused on renormalizing the physiology of micturition in UI patients without major surgery, which may lead to more normal urinary function. A variety of neuromodulation techniques have been developed, including vaginal and anal electrical stimulation (i.e., VES), percutaneous tibial nerve stimulation, electrical stimulation with implantable devices, and IVES

IVES techniques have been shown to improve UI symptoms, without adverse effects beyond rates of urinary tract infection (“UTI”) typical with other urinary catheters. IVES has been shown to enable increased bladder filling sensation, increased bladder capacity, and increased bladder compliance. IVES has also been shown enable patients to achieve more normal micturition, urinary continence and control by “retraining” the patient's micturition reflexes and nerve pathways that control the urination process.

However, while resulting generally in improvement, these studies have revealed different degrees of success in eliminating UI and restoring normal intra-bladder control, and illustrated the difficulty of determining values for stimulation treatment parameters that result in consistent and efficacious results.

B. Historical Development of VES/IVES:

M. H. Saxtorph first suggested IVES in 1878. F. Katona and others, including J. Benyo and I. Lang, contributed developments to IVES beginning in the 1950's. H. G. Eckstein and F. Katona introduced IVES in the U.S. in a Lancet research paper in 1974. W. E. Kaplan and I. Richards reported on using IVES to treat children with neurogenic bladder dysfunction in 1986. Studies of IVES over the past two decades have researched its mechanisms and its safety and effectiveness. Studies have included adults and children, both male and female, who suffered bladder dysfunction (including Stress, Urge and Mixed UI) whether by neurogenic or non-neurogenic causes.

C. Work in the Area of UI:

U.S. Pat. No. 4,569,351 describes an apparatus and method for stimulating micturition and certain muscles in paraplegic mammals by implanting a device that stimulates the sacral nerve within the spinal cord.

U.S. Pat. No. 5,704,908 describes an apparatus and method for conveying predetermined voltage pulses of a certain amplitude and duration to the inside of a patient's body cavity via electrodes positioned on the outside of an inflatable balloon inserted within that cavity.

U.S. Pat. Nos. 6,470,219, 7,306,591 B2, 8,177,781 B2, and U.S. Pat. App. No. 2012/0197247 A1 describe a system that utilizes RF energy tissue remodeling using a transurethral delivery system, including a multi-needle RF probe, which is inserted into the bladder and held in place with an inflatable balloon, then energized with RF energy to raise the tissue temperature.

Descriptions of the basic types, general causes and treatments for urinal incontinence, especially by stimulation of the sacral nerves, are provided by Leng M D, Wendy W. and Morrisroe M D, Shelby N. in “Sacral Nerve Stimulation for the Overactive Bladder,” (2006) 33 EURCNA 4 491-501, Department of Urology, University of Pittsburgh School of Medicine, 3471 Fifth Avenue, Suite 700, Pittsburgh, Pa. 15213, USA.

Descriptions of the basic types, general causes and treatments for urinal incontinence, focusing especially on Urge UI, by Swami, Satyam K. MS, MCh, FRCS, and Abrams M D, Paul, FRCS, in “URGE INCONTINENCE,” (1996) 23 EURCNA 3 417-426, Bristol Urological Institute, Southmead Hospital, Bristol, United Kingdom.

Researchers have used Sprague-Dawley rats, Wistar rats or felines as laboratoryanimal models for studying the effects of electrical stimulation methodologies, including IVES. Other clinicians have conducted certain trials with human patients.

In 1977, B. E. Erlandson and others conducted a vaginal electrical stimulation study in cats to show that urethral closure was optimized with 50 Hz pulses, while bladder inhibition was optimized with 10 Hz pulses. Erlandson concluded that the stimulation parameters should be adapted to the type or cause of incontinence. (1997) M. Fall, B-E Erlandson, T. Sundin, et.al., “Intravaginal Electrical Stimulation. Clinical Experiments on Bladder Inhibition,” Scand J Urol Nrphrol, suppl 44, 41-47.

Since 1984, W. Kaplan and I. Richards of the Chicago Memorial Children's Hospital used IVES to treat pediatric patients for UI secondary to myelodysplasia, a congenital spinal cord defect. (1986) W. E. Kaplan and I. Richards, “Intravesical transurethral bladder stimulation,” Z. Kinderchir v41. Several other hospitals also provided such IVES neuromodulation treatments for these types of patients.

More than two dozen published reports studied the clinical use of IVES in about 2,000 patients.

In 1989, H. Noto conducted an animal study and found that electrical stimulation using 50 Hz and 200 microsecond pulses increased firing on bladder post-ganglionic nerves, and that stimulation of adjacent sites in the brain inhibited bladder nerve firing.

Microcurrent Electrical Stimulation (“MES”) and Frequency Specific Stimulation (“FSS”) were first used in the 1980s by physicians in Europe and the US for stimulating bone repair in non-union fractures. There are numerous studies published on the effects of single channel microcurrent showing that it increases the rate of healing in wounds and fractures.

Microcurrent stimulation is normally applied in the range of hundreds of microamperes and it is distinct from conventional electrical stimulation. Studies have shown that microcurrent electrical stimulation can regulate the energy levels of the body by promoting production of ATP (Adenosine triphosphate) one of the principal energy sources for biochemical functions of the body. Published literature describes that microcurrents may increase ATP levels by multiples of 200-500%. Microstimulation increases energy levels in the cells, enhances blood circulation and promotes production of new cells that replace injured cells. New cells help the body to get rid of toxic substances.

In 1992, T. B. Boone conducted a small randomized clinical study on pediatric myelodysplasia patients. His study involved18 IVES and 13 control patients. Boone utilized very low stimulation current (3.2 mA) compared to most other researchers of the period (typically 10-60 mA); Boone did not report the values of other electrical parameters. Boone found no improvement of detrusor compliance or acquisition of bladder sensation in these patients. (1992) T. B. Boone, C. G. Roehrborn, and G. Hurt, “Transurethral Intravesical Electrotherapy for Neurogenic Bladder Dysfunction in Children with Myelodysplasia: a Prospective Randomized Clinical Trial”, The Journal of Urology, v148, 550-554.

In 1996, G. Kramer applied stimulations at 20 Hz and 10 mA for 90 minutes daily for a week and found generally reduced post-void residual (“PVR”), improved bladder sensation in 75% of patients, and that 19 of 35 patients using Clean Intermittent catheterization (“CIC”) could discontinue catheterization. (1996) G. Primus, G. Kramer and K. Pummer, “Restoration of Micturition in Patients with Acontractile and Hypocontractile Detrusor by Transurethral Electrical Bladder Stimulation,” Neurourol Urodyn v15, 489-497.

In 1998, S. Buyle studied 95 combinations of pulse and frequency parameters in a rat study to find optimal values at 10 Hz and 20 mS. (1998) S. Buyle, J. J. Wyandaele, K. D'Hauwers, F. Wuyts, and S. Sys, “Optimal Parameters for Transurethral Intravesical Electrostimulation Determined in an Experiment in the Rat”, European Urology, v33 no 5.

Also in 1998, CH Jiang studied IVES in rats using 20 Hz, 500 microsecond pulses for 5 minutes. Jiang chose stimulation current values to maximize bladder contractions. Jiang found that the micturition threshold volume decreased in all animal subjects after IVES. (1998) CH Jiang, “Modulation of the Micrurition Reflex Pathway by Intravesical Electrical Stimulation: An Experimental Study in the Rat,” Neurourology & Urodynamics, v17, 543-553.

In 1999, CH Jiang also showed in an animal study that stimulating bladder and urethral A-delta fibers induced micturition reflexes. These reflexes were much enhanced after repetitive stimulations using 20 Hz for 5 minutes. (1999) CH Jiang and S. Lindstrom, “Prolonged Enhancement of the Micturition in the Cat by Repetitive Stimulation of Bladder Afferents,” J Physiol (Lond) v517 no 2, 599-605.

In 2003, G. Gladh published results from treating 44 children for underactive detrusor using 20-25 Hz, 200-700 microsecond unipolar pulses, and 12-64 mA. Gladh observed improvement in both idiopathic and neurogenic patients. Gladh also found that 11 of the 15 patients using CIC were able to discontinue catheterization. (2003) G. Gladh, S. Mattsson, and S. Lindstrom, “Intravesical Electrical Stimulation in the Treatment of Micturition Dysfunction in Children,” Neurourology & Urodynamics v22, 2003, 233-242.

In 2004, M. R. Van Balken administered 5-20 Hz (up to 150 Hz) and 200-500 microsecond pulses to treat patients for bladder dysfunction. Van Balken varied the pulses up to 150 Hz and up to 1 mS while setting current (or voltage) to the maximum levels that the patients could tolerate. Van Balken found enhanced bladder sensation and detrusor contractions, and a 30-50% clinical success rate. However, Van Balken noted that his treatments considered a wide range of electrical parameter values and that he lacked a test to predict the outcome of the chosen electrical stimulation. (2004) M. R. Van Balken, H. Verguns and B. L. H. Bemelmans, “The Use of Electrical Devices for the Treatment of Bladder Dysfunction: a Review of Methods,” J Urol v172, 846-851.

In 2005, H. Madersbacher applied IVES to patients in non-neurogenic pediatric cases, in female post-surgery cases, and in elderly cases exhibiting detrusor weakness. Madersbacher used 20 Hz, 2 mS pulsewidth and 1-10 mA. After IVES, the pediatric subjects exhibited increased bladder sensation (volume detectability threshold decreasing from 300 ml to 220 ml), detrusor pressure increased from 30 cm to 54 cm H2O, and PVR decreased from 150 ml to 23 ml. In the female 1.5-12 months post-pelvic floor surgery cases, detrusor pressure increased from 6 mm to 35 mm H₂O and PVR decreased from 314 ml to 35 ml. Madersbacher observed similar improvements in the female 13-44 months post-surgery cases. In the elderly cases, detrusor pressure increased from 12 cm to 19 cm H₂O and bladder volume increased from 138 ml to 211 ml. Madersbacher also found that about one-third of the patients using CIC were able to discontinue catheterization. (2005) H. Madersbacher, G. Kiss, and D. Mair, “Bladder Rehabilitation in Neurogenic and Non-neurogenic Detrusor Dysfunction with Intravesical Electrotherapy,” Clin. Neurosci/Ideggy Szle, v. 58, no 9-10, 329-333.

In 2007, EMED Technology Corp. offered a product to treat patients, providing variable pulse characteristics and pre-programmed sets of parameters, including frequencies ranging from 5 Hz to 50 Hz, pulse widths ranging from 50-350 microseconds, and pre-programmed pulse configurations or packages of pulses and intervals.

In 2008, C. H. Hong studied the impact IVES on spinal cord injury (“SCI”) in rats. Hong found a decrease in the number of non-voiding detrusor contractions and maximal pressure of non-voiding detrusor contractions compared to sham stimulation. Hong found a decrease in the mean maximal voiding pressure compared to sham stimulation, and a significant reduction in the interval between voiding contractions compared to sham stimulation. Hong concluded that IVES significantly restored the balance between the levels of excitatory and inhibitory responses in the lumbosacral spinal cord, which acted to inhibit detrusor hyperreflexia. (2008) C. H. Hong, et.al., “The Effect of Intravesical Electrical Stimulation on Bladder Function and Synaptic Neurotransmission in the Rat Spinal Cord after Spinal Cord Injury,” BJU Intl, v103, 1136-1141.

Also in 2008, F. Katona published a review of IVES. Katona included a table of suggested electrical/pulse parameters, categorized based on the etiology of the condition and goals of the therapy. Katona suggested 70-100 Hz generally used for inhibition of the detrusor and 15 Hz generally used for relaxation of the sphincter and perineum. Katona suggested a variety of pulse risetimes, pulse intervals and configurations or packages of pulses and intervals. Katona suggested a typical duration for a treatment session of 15-90 minutes. (2008) H. G. Madersbacher, F. Katona, M. Berenyi, “Intravesical Electrical Stimulation of the Bladder,” in: Textbook of the Neurogenic Bladder 2nd Edition, Eds: J. Corcos, E. Schick, Informa UK, pp: 624-629.

Further in 2008, H. Madersbacher stated in a text book article that IVES has been shown to successfully induce and improve bladder sensation and micturition reflex. He noted, however, that controversy in the literature arises mainly due to differing inclusion or exclusion criteria used by researches when selecting study subjects and by lack of transparency in electrical stimulation parameters used. (2008) H. G. Madersbacher, F. Soldier, M. Berenyi, “Intravesical Electrical Stimulation of the Bladder,” Textbook of the Neurogenic Bladder 2nd Edition, Eds: J. Corcos, E. Schick, UK Informa, 624-629.

In 2009, F. DeBock conducted an animal study involving various electrical stimulation parameters, comprising a constant-current unipolar square wave or sawtooth pulse at 5, 10, or 20 Hz and 10, 20, or 100 mS pulsewidths. DeBock found that square waves resulted in higher maximal pressure response compared to sawtooth waveforms; however, he also found these results were correlated to the amount of charge delivered by each waveform.

In 2011, F. DeBock-2 conducted an animal study involving a variety of waveforms including unipolar square waves, biphasic square waves, asymmetric biphasic square waves, double square waves, with unipolar exponential rise, biphasic exponential rise, and double exponential rise. DeBock applied pulse durations of 5 mS or 20 MS at a frequency of 10 Hz for 5 minutes. DeBock studied the impact of the various parameters on detrusor contractions. He found the contractions exhibited the same maximal pressure rise for all waveforms with varying average power. DeBock's study also showed that charge-balanced waveforms were more comfortable for the patient compared to unbalanced waveforms, with no difference in outcome. DeBock also found that the required stimulation power to achieve equivalent results depended on the waveform selected.

Based on these studies, Table 1, below, summarizes typical ranges for electrical stimulation parameters:

TABLE 1

| PARAMETER | Stimulation parameters |
|---|---|
| Current (mA) | 3-80 |
| Voltage (V) | 0-80 |
| PulseWidth | 0-1000 (one thousand) |
| (microseconds) | |
| Frequency (Hz) | 1-150 |
| Pulse type | Biphasic and other |
| Pulse balance | Symmetrical (no DC component) |
| Pulse shape | Rectangular and other waveforms as |
| | needed |

Thus, an understanding of IVES therapies has evolved over the past few decades, and numerous studies involving IVES and its treatment protocols have been conducted. While various IVES studies have generally demonstrated improvements in patients with UI, these studies have resulted in a range of efficacies in treating UI and restoring intra-bladder control. IVES has been shown to improve the overall symptoms of UI by providing beneficially increased bladder filling sensation, increased bladder capacity, and increased bladder compliance. IVES has also been shown to “retrain” the patient's micturition reflexes and nerve pathways that control the urination process, enabling the patient to achieve more normal micturition, urinary continence and control. Despite the studies and research that have been done, there is still a need for determining values of stimulation treatment parameters that result in consistent and efficacious results in eliminating UI and restoring normal intra-bladder control.

While IVES therapies have shown positive results, their efficacy remains to be optimized.

What is needed is a system and method for treating UI that is both effective and low in risk. What is further needed is an effective system and method of treating UI and related conditions using various therapies comprised of improved neuromodulation techniques and intravesical electrical stimulation (“IVES”).

For purposes of the present application, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned. While certain aspects of conventional technologies have been discussed to facilitate disclosure of the invention, applicants in no way disclaim these technical aspects, and it is contemplated that the claimed invention may encompass one or more of the conventional technical aspects discussed herein.

### BRIEF SUMMARY OF THE INVENTION

It is accordingly an object of the invention to provide an inventive system and method of treating UI and related conditions using various therapies comprised of improved neuromodulation techniques and intravesical electrical stimulation (“IVES”). In one particular embodiment of the invention, feedback is used to create a closed-loop system for iteratively adjusting the IVES treatment provided to a patient.

In another embodiment of the invention, enhanced sensors are used in combination with a biofeedback, to provide a closed-loop system with signal processing, and algorithms to modify stimulation parameters based on a patient's responses. The utilization of biofeedback as well as the integration of biological sensors to complete a closed-loop system operation may significantly enhance the clinical benefits of IVES. In one particular embodiment of the invention, the integration of multiple sensors that react to specific patient response and dynamic requirements facilitates the customization of stimulation algorithms to adjust to the dynamics of the response.

In one particular embodiment, a closed-loop system optimizes IVES therapy by retro feeding information about the patient and his responses to determine treatment values in subsequent therapy sessions. In another embodiment, therapy parameters are optimized over time to improve the efficacy of the IVES treatment. Certain embodiments of the present invention include, but are not limited to, a multi-module closed-loop system.

Certain embodiments of the present invention include, but are not limited to, multiple therapy modalities used as treatment options to enhance patient results and therapy efficacy, including, but not limited to, the combination of IVES, VES, Surface Electrical Stimulation (“SES”), stimulation by implanted electrodes, and other approaches to maximize patient response.

Certain embodiments of the present invention include, but are not limited to, a system and method providing IVES and related electrical stimulation therapy, optimizing treatment parameters, and incorporating multiple treatment modalities under a comprehensive closed-loop system to maximize therapeutic efficacy of the treatment. In an embodiment, neurological modulation therapy is directed to patients experiencing conditions of Urge UI, Stress UI, Mixed UI, Neurophatic, and other forms or degrees of UI, or other conditions resulting from lack of normal control of the bladder system. Certain embodiments of the present invention include, but are not limited to, an electrical stimulation module, stimulation probes/catheters, and a measurement and feedback system that determines an initial and subsequent electrical stimulation therapy modality as a function of various inputs, including, but not limited to, a pre-programmed library and measured and patient-provided response data. In an embodiment, the system generates the IVES and other electrical stimulation signals and modalities and conveys the signals and modalities via electrodes placed in and around the bladder system.

Certain embodiments of the present invention include, but are not limited to, an ESM that generates the electrical treatment parameters comprising various treatment modalities involving IVES techniques. Modalities include, but are not limited to, IVES alone or in combination with one or more of VES, surface electrical stimulation around the pelvic floor, urethral, rectal, and other various surface (SES) or implanted electrodes. The ESM determines electrical treatment parameters and modalities to stimulate the patient's afferent neurological pathways in a way similar to the body's natural micturition feedback mechanism, hence re-training the bladder system to achieve a normal “bladder-sphincter equilibrium.”

Certain embodiments of the present invention include, but are not limited to, electrical safety features including, but not limited to, safety algorithms to clamp voltages and currents within safety thresholds, a one-time use catheter connection to prevent unsanitary re-use of the catheter, and catheter electrical-shock protections to ensure that electrode conductors do not make unintended contact with sensitive bodily tissues.

In an embodiment, the electrode configuration within the catheter ensures accurate and safe placement of the conductors within the bladder system to provide sufficient surface contact area with the intra-bladder liquid while not touching the inner tissues of the bladder.

Certain embodiments of the present invention include, but are not limited to, a method of a physician using the system to administer electrical stimulation treatments to a patient. In an embodiment, at initialization the physician considers the patient's condition and selects preconfigured electrical stimulation parameters specified by the historical clinical result tabulations stored in the MM. Following initialization, an embodiment utilizes a closed-loop process as part of the feedback response mechanism to adjust the electrical stimulation parameters. The closed-loop process calculates optimized parameter values as a function of various input parameters including, but not limited to, parameters measured by the FIM, combined with patient-specific feedback responses collected by the RFM and collated by the RSM, patient-specific measured parameters stored in the MM, and historical clinical results stored in the MM. Based on these inputs, the CPM executes algorithms to determine electrical stimulation parameters to deliver the next treatments cycle to the patient.

Certain embodiments of the present invention include, but are not limited to, a system for providing electrical neuromodulation treatment, comprising: an Electrical Stimulation Module, comprising: a means for receiving an at least one Stimulation Parameter Input; a means for determining an at least one Stimulation Parameters Output Group; and a means for conveying said at least one Stimulation Parameters Output Group comprising IVES.

Certain embodiments of the present invention include, but are not limited to, a system for providing electrical neuromodulation treatment, comprising: an Electrical Stimulation Module, comprising: a means for receiving an at least one Stimulation Parameter Input; a means for determining an at least one Stimulation Parameters Output Group; and a means for conveying said at least one Stimulation Parameters Output Group comprising IVES and microcurrent stimulation.

Certain embodiments of the present invention include, but are not limited to, a system for providing electrical neuromodulation treatment, comprising: an Electrical Stimulation Module, comprising: a means for receiving an at least one Stimulation Parameter Input; a means for determining an at least one Stimulation Parameters Output Group; and a means for conveying said at least one Stimulation Parameters Output Group comprising IVES.

Certain embodiments of the present invention include, but are not limited to, a system for providing electrical neuromodulation treatment, comprising: an Electrical Stimulation Module, comprising: a means for manually receiving an at least one Stimulation Parameter Input; a means for determining an at least one Stimulation Parameters Output Group, a means for conveying said at least one Stimulation Parameters Output Group comprising IVES.

Certain embodiments of the present invention include, but are not limited to, a catheter, comprising: a means for conveying an at least one Stimulation Parameters Output Group comprising IVES; an at least one orifice permitting fluidic contact between said at least one means for conveying said at least one Stimulation Parameters Output Group and intra-bladder fluid; and a non-conductive mesh lining the inner circumference of an at least one lumen of said catheter aligned beneath said at least one orifice of said at least one lumen of said catheter.

Certain embodiments of the present invention include, but are not limited to, a catheter, comprising: a means for conveying an at least one Stimulation Parameters Output Group comprising IVES; at least one orifice permitting fluidic contact between said at least one means for conveying said at least one Stimulation Parameters Output Group and intra-bladder fluid; and at least one rib that rings the inner circumferential surface of an at least one lumen of said catheter; said at least one rib having a thickness dimension in the radial direction that extends from the outer surface of said means for conveying said at least one Stimulation Parameters Output Group to the inner surface of said at least one lumen of said catheter; said at least one rib having a width dimension substantially identical to the thickness dimension of said at least one rib; and said at least one rib positioned and aligned adjacent to said at least one orifice of said at least one lumen.

Certain embodiments of the present invention include, but are not limited to, a catheter comprising a one-time connector comprising: a rigid connector housing having a top, bottom, front, back left side and right side surface; a housing channel lumen vertically formed within said rigid connector housing, extending between the top surface to the bottom surface of said rigid connector housing and positioned between the rear inner surface and the front inner surface of said rigid connector housing, said housing channel lumen comprising an axial channel having a diameter substantially sized to receive an input conductive wire connector entering within said axial channel at the top aperture of said axial channel, and to receive an output conductive wire plug connector entering within said axial channel at the bottom aperture of said axial channel; a lock pin chamber comprising a cavity horizontally formed within said rigid connector housing and positioned between the top inner surface and the bottom inner surface of said rigid connector housing, said lock pin chamber further comprising a diameter substantially equal to said diameter of said housing channel lumen, said lock pin chamber extending between the back surface to the front surface of said rigid connector housing, in the direction tranverse to and intersecting with said housing channel lumen; a compressed spring positioned within said lock pin chamber adjacent to the back surface of said rigid connector housing, said spring exerting a restoring force in the direction parallel to the axial channel of said lock pin chamber and toward the front of said rigid connector housing; a lock pin comprising a rigid plug positioned adjacently to said spring and slideably mounted and guided within said lock pin chamber, said lock pin comprising a length greater than said diameter of said housing channel lumen; a barrier pin lock comprising a rigid hollow cylinder axially aligned and formed within said housing channel lumen, said barrier pin lock comprising a height shorter than the distance between the bottom of said housing to the bottom of said lock pin chamber, said barrier pin lock further comprising a second axial channel having a diameter sized to receive an input connector entering within said second axial channel at the top aperture of said second axial channel, and to receive an output conductive wire plug connector entering within said second axial channel at the bottom aperture of said second axial channel; said barrier pin lock further comprising a first locking ridge feature positioned on the inner surface of said barrier pin lock within said barrier pin lock substantially near the top of said barrier pin lock and a second locking ridge feature positioned on the inner surface of said barrier pin lock substantially near the bottom of said barrier pin lock, said first and second locking ridge features comprising a distance between the two measured in the axial direction of the housing channel lumen such that said distance is substantially equal to the diameter of said housing channel lumen, said locking ridge features each further comprising an inner diameter thickness measured radially in the inward direction from the inner surface of said barrier pin lock to the inner surface of said inner diameter, said inner diameter sized to block said input connector from entering said inner diameter and to receive said output conductive wire plug connector entering within said inner diameter, said first and second locking ridge features each further comprising a top surface formed in the downward slanting direction with a substantially obtuse angle measured from the direction parallel to the inner vertical surface in the upward direction of said barrier pin lock, said first and second locking ridge features further comprising a bottom surface formed with a substantially horizontal surface that is substantially orthogonal to the inner vertical surface of said barrier pin lock; and a barrier pin comprising a rigid hollow cylinder axially aligned and formed within said barrier pin lock, said barrier pin comprising a height shorter than the height of said barrier pin lock, said barrier pin further comprising a third axial channel having a diameter sized to block said input connector from entering said third axial channel at the top aperture of said third axial channel, but to receive said output conductive wire plug connector entering within said third axial channel at the bottom aperture of said third axial channel, said barrier pin further comprising a tensile semi-rigid lip extending in the outward radial direction and positioned at the bottom of said barrier pin, said lip having a width relative to the length of said first and second locking ridge features that is substantially sufficient to move unrestrictedly past said locking ridge feature when said barrier pin moves in the downward direction but to catch and stop against said locking ridge feature when said barrier pin moves in the upward direction.

Certain embodiments of the present invention include, but are not limited to, a method of electrical neurostimulation treatment to the bladder-system area of a patient comprising steps of: selecting initial Baseline Stimulation Parameters to apply timed electrical pulses of varying characteristics across electrodes positioned in said bladder-system area of said patient; and selecting an automatic mode for determining subsequent Stimulation Parameters to apply said timed electrical pulses.

Certain embodiments of the present invention include, but are not limited to, a method of electrical neurostimulation treatment to the bladder-system area of a patient comprising steps of: selecting initial Baseline Stimulation Parameters to apply timed electrical pulses of varying characteristics across electrodes positioned in said bladder-system area of said patient; storing information measured at said electrodes following application of said timed electrical pulses; and selecting an automatic mode for determining subsequent Stimulation Parameters to apply said timed electrical pulses.

Certain embodiments of the present invention include, but are not limited to, a method of electrical neurostimulation treatment to the bladder-system area of a patient comprising steps of: selecting initial Baseline Stimulation Parameters to apply timed electrical pulses of varying characteristics across electrodes positioned in said bladder-system area of said patient; selecting an automatic mode for determining subsequent Stimulation Parameters to apply said timed electrical pulses; and adjusting said electrodes within said bladder-system area of said patient in response to the requirements of said automatic mode for determining said subsequent Stimulation Parameters.

Although the invention is illustrated and described herein as embodied in an electrical neuromodulation stimulation system and method for treating urinary incontinence, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

For the purpose of illustrating the invention, there is shown in the drawings an exemplary embodiment that is presently preferred, it being understood however, that the invention is not limited to the specific methods and instrumentality's disclosed. Additionally, like reference numerals represent like items throughout the drawings. In the drawings:

FIG. 1A is a diagram illustrating a simple reflex loop model of normal urinary inhibition;

FIG. 1B provides a more detailed view of the process of volitional micturition;

FIG. 1C is a table summarizing many of the common UI therapies, an approximate degree of invasiveness, and range of efficacy and complications commonly reported in the literature.

FIG. 2A depicts a process flow diagram of a system in accordance with one particular embodiment of the present invention;

FIG. 2B is a block diagram of a system in accordance with one particular embodiment of the present invention;

FIG. 3 depicts a perspective view of a system in accordance with one particular embodiment of the present invention;

FIG. 4 depicts a schematic overview diagram of the system in accordance with one particular embodiment of the present invention;

FIG. 5 is a flowchart illustrating one particular embodiment of a stimulation determination algorithm;

FIG. 6 depicts an exploded view of a catheter and assembly in accordance with one particular embodiment of the present invention;

FIG. 7 depicts an exploded view of one particular embodiment of a one-time use connector for use with the catheter and assembly of FIG. 6;

FIG. 8 depicts a side cutaway sectional view of barrier pin lock of the one-time use connector of FIG. 7;

FIG. 9 depicts a sideward facing elevation view of barrier pin of the one-time use connector of FIG. 7;

FIG. 10 depicts a sideward facing elevation view of barrier pin of FIG. 9;

FIG. 11 depicts a downward facing elevation view of lock pin of the one-time use connector of FIG. 7;

FIG. 12 depicts a rearward facing elevation view of lock pin of the one-time use connector of FIG. 7;

FIG. 13 depicts a rightward facing cross sectional view of lock pin of FIG. 12 at Section L-L;

FIG. 14A depicts a side elevation view of one-time use connector, unlocked position (barrier pin in up-position) in accordance with one particular embodiment of the invention;

FIG. 14B depicts an enlarged, cross sectional view taken at Section A-A of the one-time use connector, unlocked position (barrier pin in up-position) of FIG. 14A;

FIG. 15A depicts a side elevation view of one-time use connector, locked position with connector inserted (barrier pin in up-position) in accordance with one particular embodiment of the invention;

FIG. 15B depicts a cross sectional view taken at Section A-A of the one-time use connector, locked position with connector inserted (barrier pin in up-position) of FIG. 15A;

FIG. 16A depicts a side elevation view of one-time use connector, locked position (barrier pin in down-position) in accordance with one particular embodiment of the invention;

FIG. 16B depicts a cross sectional view taken at Section A-A of the one-time use connector, locked position (barrier pin in down-position) of FIG. 16A;

FIG. 17 depicts a side elevational view of a catheter in accordance with one particular embodiment of the invention;

FIG. 18 depicts a downward looking cutaway sectional view taken at Section A-A of the catheter of FIG. 17;

FIG. 19 depicts a side elevation view of a catheter with a conductive wire helix in accordance with one particular embodiment of the invention;

FIG. 20 depicts a downward looking cutaway sectional view at Section B-B of a catheter with a conductive wire helix of FIG. 19;

FIG. 21 depicts a side elevation view of a catheter with co-extruded conductive wires in accordance with one particular embodiment of the invention;

FIG. 22 depicts a backward looking cross sectional view taken at Section C-C of the catheter with co-extruded conductive wire of FIG. 21;

FIG. 23 depicts a side elevation view of a catheter with a conductive wire mesh in accordance with one particular embodiment of the invention;

FIG. 24 depicts a downward looking cutaway sectional view taken at Section D-D of the catheter with a conductive wire mesh of FIG. 23;

FIG. 25 depicts a backward looking cross sectional view taken at Section E-E of the catheter with a conductive wire mesh of FIG. 23;

FIG. 26 depicts a side elevation view of a catheter with multi-lumens and co-extruded multiple conductive wires in accordance with one particular embodiment of the invention;

FIG. 27 depicts a backward looking cross sectional view taken at Section K-K of the catheter with multi-lumens and co-extruded multiple conductive wires of FIG. 26;

FIG. 28 depicts a side elevation view of a catheter with multi-lumens and multiple conductive wires in accordance with another embodiment of the invention;

FIG. 29 depicts a backward looking cross sectional view taken at Section J-J of the catheter with multi-lumens and multiple conductive wires of FIG. 28;

FIG. 30 depicts a downward looking cross sectional view of a protection device for IVES orifices—plastic mesh in accordance with one particular embodiment of the invention;

FIG. 31 depicts a backward looking cross sectional view taken at Section T-T of the protection device for IVES orifices—plastic mesh of FIG. 31;

FIG. 32 depicts a downward looking cross sectional view of a protection device for IVES orifices—ribs in accordance with one particular embodiment of the invention;

FIG. 33 depicts a backward looking cross sectional view taken at Section R-R of the protection device for IVES orifices—ribs of FIG. 32;

FIG. 34 depicts a downward looking cross sectional view of a protection device for IVES orifices—balloon in accordance with one particular embodiment of the invention;

FIG. 35 depicts a downward looking cross sectional view of a protection device for IVES orifices—perforated orifices;

FIG. 36 depicts a side elevation view of a catheter with an inflatable balloon electrode at the tip in accordance with one particular embodiment of the invention;

FIG. 37 depicts a downward looking cutaway view taken at Section F-F of the catheter with an inflatable balloon electrode at the tip of FIG. 36;

FIG. 38 depicts a side elevation view of a catheter with a mid-position inflatable balloon electrode in accordance with one particular embodiment of the invention; and

FIG. 39 depicts a downward looking cutaway sectional view taken at Section H-H of the catheter with a mid-position inflatable balloon electrode of FIG. 38.

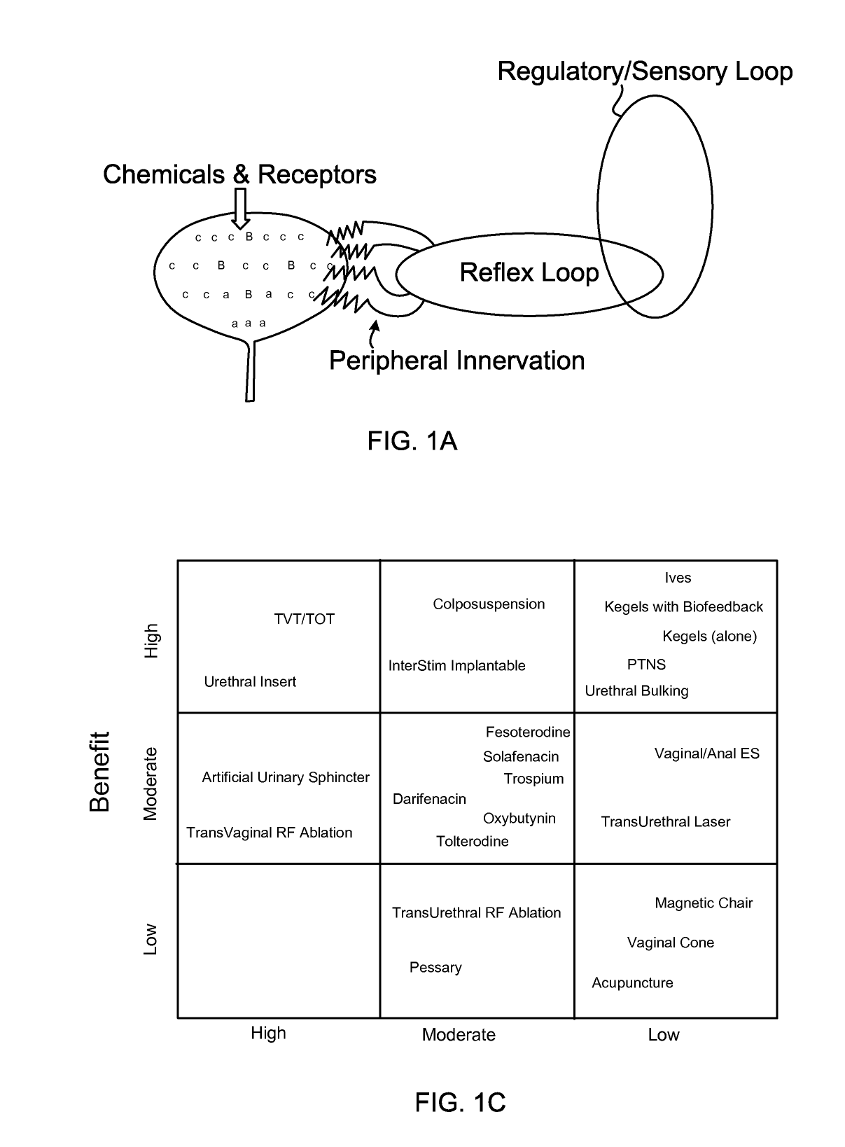

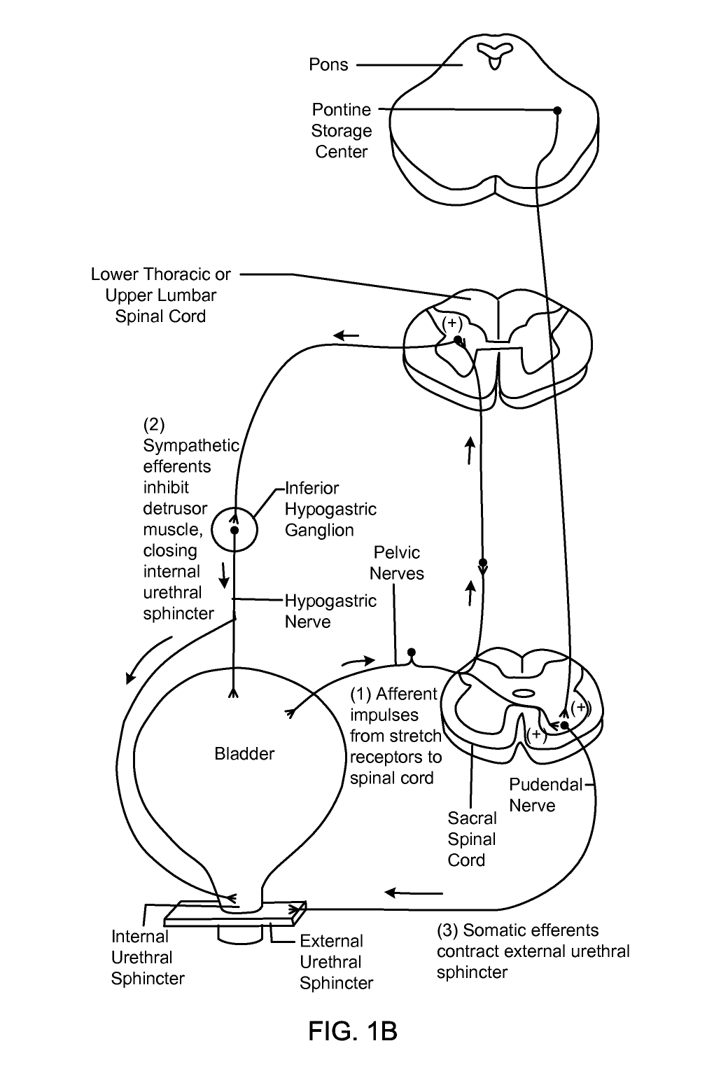

### DETAILED DESCRIPTION OF THE INVENTION

In the following description, for purposes of explanation, numerous specific details are set forth in order to provide an understanding of various embodiments of the inventive subject matter. It will be evident, however, to those skilled in the art that embodiments of the inventive subject matter may be practiced without these specific details. In general, well-known structures and techniques have not been shown in detail.

As used herein, the term “or” may be construed in either an inclusive or exclusive sense. Similarly, the term “exemplary” is construed merely to mean an example of something or an exemplar and not necessarily a preferred or ideal means of accomplishing a goal. Additionally, although various exemplary embodiments discussed below focus on verification of experts, the embodiments are given merely for clarity and disclosure. Alternative embodiments may employ other systems and methods and are considered as being within the scope of the present invention.

Reference in the specification to “one embodiment”, “one particular embodiment” or “an embodiment” means that a particular feature, structure, or characteristic described in connection with the embodiment is included in at least one embodiment of the invention, but not necessarily only one embodiment. Similarly, the use of the phrases “in one embodiment” or “in one particular embodiment” in various places in the specification are not necessarily all referring to the same embodiment, but rather, could relate to the same embodiment or different embodiments.

In the description that follows, any reference to either orientation or direction is intended primarily and solely for the purpose of illustration and is not intended in any way as a limitation of the scope of the present invention or its claims. Also, the particular embodiments described herein although being noted as preferred are not to be considered as limiting of the present invention. Furthermore, like-parts or like-elements in the various drawings hereto are identified by like-numerals.

System Architecture Diagram:

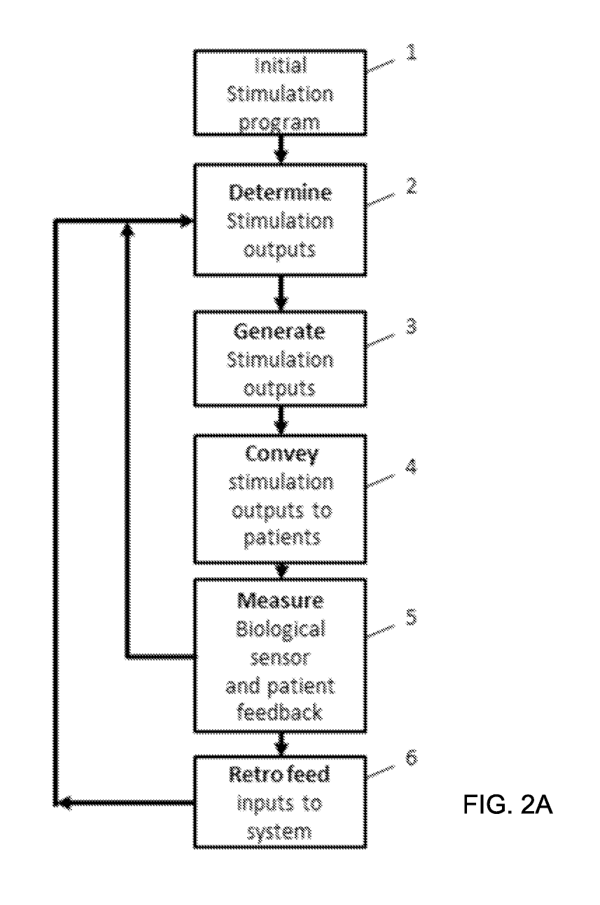

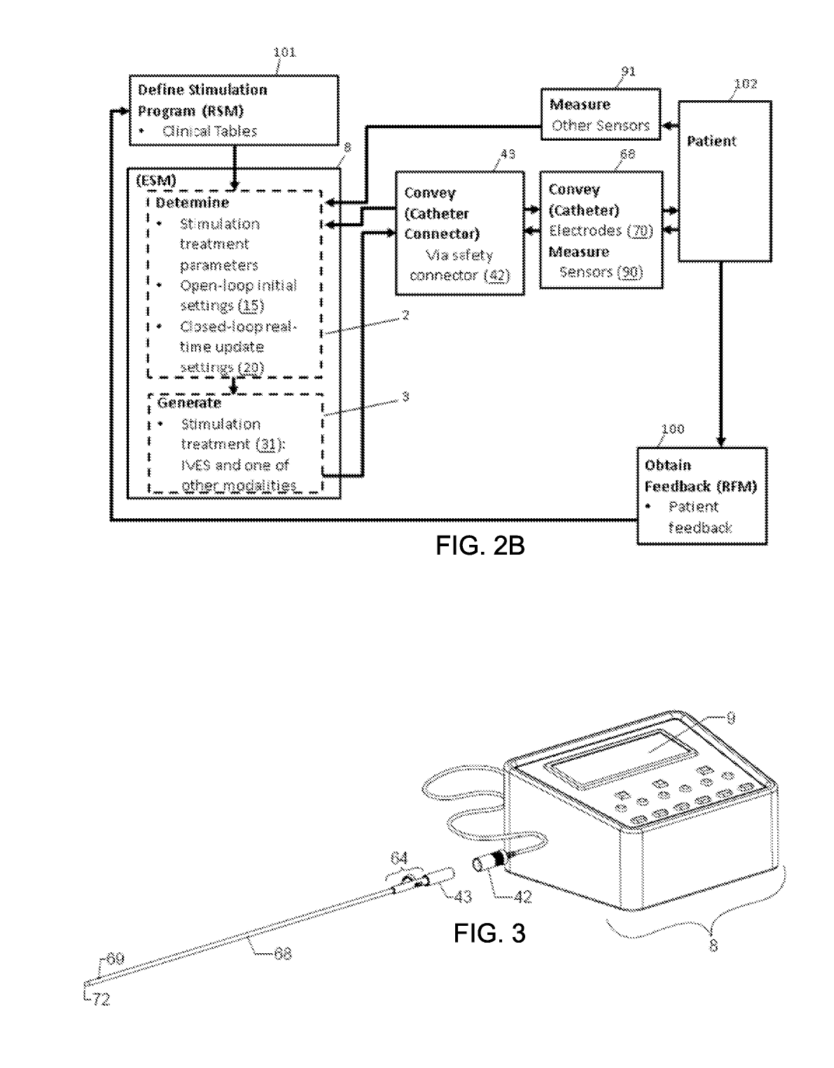

FIG. 2A illustrates a method in accordance with one particular embodiment of the present invention that includes, but is not limited to, a conceptual process flow as indicated. In the present embodiment, the system first initializes system software and system parameters. Step 1. Next, the system determines appropriate parameter values (“Stimulation Parameters”) corresponding to a stimulation treatment therapy session, including, but not limited to, values representing voltage, current, pulse width, frequency, waveform shape, and waveform phase. Step 2. In step 3, the system generates the electrical levels to administer the stimulation treatment (“Stimulation Parameters Output Group”) to a patient (102 of FIG. 2B) during the stimulation treatment session. Subsequently, the system conveys the Stimulation Parameters Output Group to the patient. Step 4. Following the administration of the therapy session, the system measures responses from the patient via biological sensors and patient-generated feedback. Step 5. Subsequently, in step 6, the responses measured in step 5 are fed back to the system (i.e., via a “retro feed”) for subsequent use in a feedback calculation loop to determine stimulation outputs for the particular patient (step 2). In one particular embodiment, trend analysis including, but not limited to, the patient's pre-treatment condition, the reaction of the patient to the specific parameters of the therapy as well as signal analysis of the various points of the bladder system are utilized to provide proper feedback for subsequent parameter computation including, but not limited to, specific spectrum of frequencies, time frame for alternation, and corresponding intensity during each frequency period.

System Block Diagram:

FIG. 2B shows one particular embodiment of a system abstraction block diagram of components for performing the process steps described above in FIG. 2A. Referring now to FIGS. 2A and 2B, in the presently described embodiment, a remote server module (RSM) 101 centrally stores information and makes available for downloading system initialization inputs in the form of prior stimulation results and tabular data of clinical stimulation parameter history. In one embodiment, the RSM 101 collects patient response information and clinician inputs, including, but not limited to, the timing of the patient's micturition cycle and survey questionnaire responses regarding the patient's bladder system and observed clinical results. The RSM 101 is coupled to an electrical stimulation module (ESM) 8 via a, network (e.g., LAN, WAN, etc.), Internet or other data connection. ESM 8 determines the stimulation treatment parameters (step 2) either in an open-loop mode 15 or a closed-loop mode 20, and generates the stimulation treatment 31 (step 3), including, but not limited to, electrical output levels and signals directed to intravesical electrostimulation (IVES) and other treatment modalities. The use of a closed-loop mode 20 can optimize the selection and delivery of concurrent multiple stimulation signals and modalities to treat more than one specific condition.

A catheter connector 43, including, but not limited to, a plug or safety connector 42, electrically conducts the electrical levels and signals from the ESM 8 to electrodes 70 formed in a catheter 68, which is inserted in the patient 102. In addition to conveying the stimulation treatment, the catheter 68 includes sensors 90 positioned in the catheter 68 to measure electrical levels at the patient 102. For example, sensors 90 embedded in the catheter provide sensor inputs to the ESM 8, including, but not limited to, measurements of bladder system voltage and current. Other measurement sensors and electrodes 91 can be provided in the catheter 68, or elsewhere on or about the patient, to measure biological reactions of the patient. Other sensory inputs provided via standard connectors to the ESM 8 include, but are not limited to, vaginal pressure sensors, urethral pressure sensors, intra bladder pressure sensors, bladder-system electromyography sensors, bladder volume measurement sensors, urethral closure sensors, a residual urine volume sensor, and biological sensors. Biological sensors are those of the body involved in adjusting function of the bladder system, which include, but are not limited to, meridian voltage points, bladder mucosa, mechanoreceptors, somatic innervations and others.

Measured and calculated parameters include, but are not limited to, the bladder system's impedance, Electroencephalography (“EEG”) and bladder-system electromyography (“EMG”). Biological sensory information includes, but is not limited to, vaginal, sphincter and intravesical pressure measurements, bladder compliance, and residual urine measurements. The catheter 68 may also be used in conjunction with SES electrodes or implanted stimulating electrodes.

Table 2, below, summarizes exemplary measured and sensory and calculated feedback parameters that may be used in particular embodiments of the present invention:

TABLE 2

| | | |
|---|---|---|
| | SENSOR | |
| PARAMETER | DESCRIPTION | LOCATION/REMARKS |

ELECTRICAL SIGNALS

| | | |
|---|---|---|
| Bladder | (No sensor). | Voltage and current measurements with Ohms law. |
| system's | Determined | Differential measurements by external sensors |
| impedance | based on | comprising two electrodes on the bladder wall based |
| | voltage and | on the first emitting and the second receiving an |
| | current of the | electrical signal. |
| | stimulation | Cystogram (off line). |
| | output signal, | Correlation between sensors and cystograms. |
| | measured by | |
| | catheter | |
| | electrodes as | |
| | measured | |
| | parameters. | |
| EEG of | Surface | Placed on specific nerves. |
| control | electrodes on | Muscles require electricity to contract. This electricity |
| signals | nerves coming | comes from nerves in the spinal cord which originate |
| | from the brain | in the brain. With age and/or injury the power output |
| | (efferent | can be decreased. |
| | signals). | Analysis of the control signals (EEG) and correlation |
| | | between expected output with actual bladder system |
| | | performance (bladder, sphincters, urethra, spinal |
| | | cord, pelvic, sacral, Pudental nerve and others) is |
| | | helpful in determining stimulation required. |
| EMG of | Surface | Abdominal, vaginal, pelvic floor and surrounding |
| various | electrodes on | muscle structures are the principal points where EMG |
| muscles | muscles | signals are monitored. |
| associated | | Synchronization of signals as well as proper |
| with the | | mechanical sequences are key indicators of |
| bladder | | diagnosis and thus can determine required |
| system | | stimulation. |

PRESSURE SIGNALS

| | | |
|---|---|---|
| Vaginal | Perionometer | Detruset or special probe. |
| pressure | | Vaginal pressure can be correlated to bladder re- |
| | | education. |
| Intravesical | Pressure sensor | Detruset or special probe. |
| pressure | | A cystogram permits determination of various |
| | | pressures: Intravesical, Detrusor and Abdominal. |
| Urethral | Pressure | Detruset or special probe. |
| Sphincter | sensors | Urethral pressures can be measured at individual |
| pressure | | locations within the urethra (point pressures) or along |
| | | the whole length of the urethra. Values at different |
| | | bladder volumes and patient conditions (during |
| | | coughing, resting, voiding and others) can contribute |
| | | to a more precise diagnostic. |
| | | Two micro transducers enclosed in the catheter can |
| | | be used to record urethral pressure profile |
| | | simultaneously with intravesical pressure. Detailed |
| | | information about normal micturition as well as stress |
| | | and urgency incontinence can be obtained. The |
| | | functional as well as the absolute length of the |
| | | urethra can be estimated within half a millimeter. |
| Urethral | Proximity | Detruset or special probe. |
| Sphincter | sensor | The simultaneous recording of both urethral (P u) and |
| closure | | intravesical (P i) pressure enables calculation of |
| | | urethral closure pressure (P c), where P c = P u − P i. |
| | | The balloon method involves a cylindrical balloon |
| | | mounted concentrically on a catheter. The balloon |
| | | requires pressure of only a few centimeters of water |
| | | to be inflated to its maximum diameter. |
| | | A balloon that is long in comparison with the axial |
| | | distances tends to average out differences in |
| | | pressure along the length of the urethra as well as |
| | | pressure variations. |
| Bladder | | Bladder Compliance describes the relationship |
| compliance | | between change in bladder volume and change in |
| | | detrusor pressure and is defined as DV/DP. |
| | | The rise in pressure that causes lower compliances is |
| | | a function of the viscoelastic nature of the detrusor |
| | | under higher filling rates, i.e. stretch the muscle too |
| | | fast and it cannot accommodate completely. |
| | | Specific bladder condition is a function of age or |
| | | neurological condition and its status can be an |
| | | important indication of dysfunction and improvement. |

VOLUME SIGNALS

| | |
|---|---|
| Residual | Residual urine can be an indication of bladder |
| urine | dysfunction. It can be measured ultrasonically and |
| | with catheterization. Typically this measurement is |
| | performed at a time when stimulation is not being |
| | administered. |

Measurements from the sensors 90, 91 are conveyed back to the ESM 8. Additionally, a remote feedback module (RFM) 100 receives feedback information from the patient 102 regarding the treatment, collates the feedback responses, and sends the information back to the RSM 101 for storage, via a Network, Internet or other data connection.

Referring now to FIG. 3, there is shown one particular embodiment of a device for use in one particular embodiment of the present invention. More particularly, FIG. 3 is a perspective view of one particular embodiment of an ESM 8, comprising a user interface module (UIM) 9, a length of conductor terminating in a plug connector 42 that connects the ESM 8 to a connector 43 of the catheter assembly 68. The UIM 9 provides an interface with which the treatment provider can interact, for example, to select a treatment modality or to input patient feedback. Additionally, if desired, the UIM 9 can be provided with a simplified display and buttons to facilitate usage of the ESM 8 by the patient at home, as opposed to within the doctor's office or as an out-patient procedure. In one particular embodiment of the invention, the UIM 9 includes a display device and a keyboard or other data input interface, as known.

The catheter 68 of the present embodiment includes a Y-connector 64, a catheter orifice 69 towards the proximal end of the elongate body, and a catheter tip 72 at its proximal end (i.e., most proximal to the patient/point first inserted into the patient). Note that other catheter designs could be used without departing from the scope or spirit of the present invention. However, in the present configuration, the electrode can utilize of the conductive properties of urine or injected saline within the bladder system, which is inherent to IVES.

Electrical Stimulation Module (ESM) 8:

In accordance with one particular embodiment of the invention, the ESM 8 calculates an electrical stimulation therapy modality as a function of inputs including, but not limited to, a pre-programmed library. Following an electrical stimulation treatment, an embodiment's closed loop feedback mechanism processes various inputs, including, but not limited to, measuring electrical parameters, collecting biological sensory information, downloading patient response information provided by the patient via a remote server, incorporating clinician input during therapy sessions, and retro feeding the parameters, information and inputs back into the system.

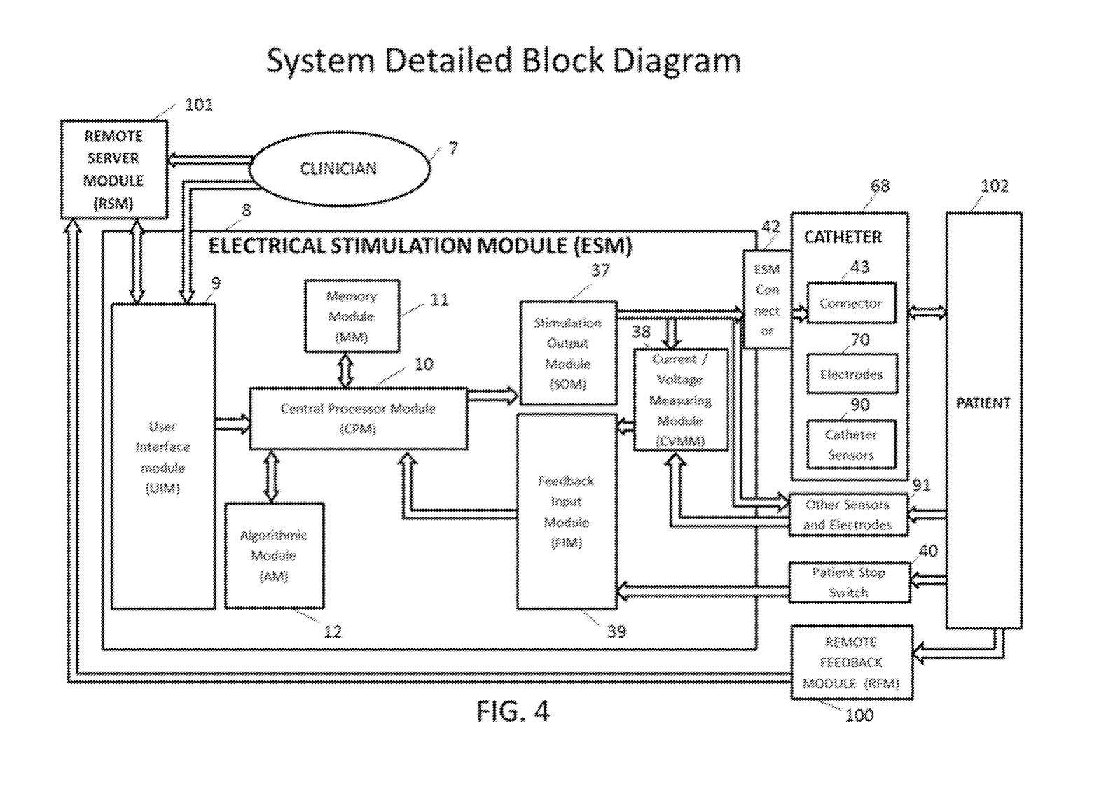

Referring now to FIGS. 2A-4, one particular embodiment of the invention will be described in greater detail. As illustrated in FIG. 4, a clinician 7 interacts with (i.e., uses) the ESM 8 to provide a treatment to a patient 102. In the present particular embodiment, the ESM 8 is illustrated as including: a user interface module (UIM) 9 to route input signals from the patient or doctor; a central processing module (CPM) 10 to control the system; a memory module (MM) 11 to store historical tabulated clinical results and updated response values; an algorithmic module (AM) 12 that executes various calculations; a stimulation output module (SOM) 37 to generate the output treatment Therapy Modality; a current voltage measuring module (CVMM) 38 and feedback input module (FIM) 39 to transform input measurement signals into usable logic signals and properly route them; an ESM connector 42 and a patient stop switch (PSS) 40. Note that the ESM 8 is not meant to be limited to only those parts enumerated in FIG. 4, but rather, more or fewer components can be used without departing from the scope of the present invention.

In the ESM 8, the CPM 10 can include at least one of a processor, a microcontroller, a hard-wired circuit, an ASIC, an FPGA, or another logic device that is particularly configured to execute a method of the present invention. In one particularly preferred embodiment of the invention, the CPM 10 is a processor that executes a program or algorithm stored in non-transitory fashion in the MM 11, which configures the CPM to perform the methods described in connection with the present invention. Directional arrows in FIG. 4 illustrate the primary direction of electrical signal-bus communication connections between the elements of the system of that embodiment. Calculation algorithms executed by the AM 12 in conjunction with the MM 11 under control of the CPM 10, include, but are not limited to, calculating optimized output stimulation values as a function of input stimulation parameter values, the patient's measured responses resulting from prior administrations of a treatment, and historical clinical result tabulations. Algorithms also include calculating bladder compliance and bladder system impedance as a function of various measured response values including current and voltage.

As additionally shown in FIG. 4, other components are electrically connected to the ESM 8, including, but not limited to, a catheter 68 (i.e., connected via an ESM or plug connector 42 and a catheter connector 43), the catheter electrodes 70 and the catheter sensors 90. Other sensors and electrodes 91 not contained in the catheter 68 can additionally provide information (i.e., measured biofeedback of the patient), in the form of electrical signals back from the patient 102 to the ESM 8. The plug or ESM connector 42 is an electrically conductive wire connector that electrically connects the ESM 8 to the catheter connector 43. In the present particular embodiment illustrated in FIG. 4, for safety, a PSS 40 is electrically connected to the ESM 8 (either on the ESM 8 or connected to the ESM8 via a wired switch or pushbutton proximal to the patient 102), and can be pressed by the patient 102 to initiate an emergency stop condition during treatment that disables electrical output signals based on the manual intervention by the patient. The PSS 40 facilitates safe usage of the ESM 8 by the patient whether at home or at the doctor's office.

In FIG. 4, the patient is represented by block 102. In practice, a clinician 7 physically inserts the catheter 68 into the patient's treatment area, such as the urethra, bladder, vagina, anus, or area of the perineum, and/or places other sensors and electrodes 91 on or in the patient adjacent to other nerves, nodes and motor control points related to the bladder system and its peripheral and central control. Additionally as illustrated in FIG. 4, an RFM 100 (which interfaces to the patient 102) can be provided in order to convey patient feedback to the RSM 101 and the ESM 8, via a data connection between the RFM 100 the RSM 101, and a data connection between the RSM 101 and the ESM 8.

User Interface Module 9

As shown more particularly in FIG. 4, it is planned that a clinician 7 will interact with the ESM 8 through the UIM 9. In one particular embodiment, the UIM 9 comprises a simple interface panel including, but are not limited to, selector buttons, an input numeric keypad, and a display device, such as LED numeric readouts, LCD displays and/or touch screens. Alternative forms of user-inputs are additionally contemplated within the scope of the present invention, such as pushbutton, key-in, dial-in, mouse or pointer-device selected, voice activated controls, or finger-swiped inputs.

In one particular embodiment, a selector button is used by a clinician 7 to indicate to the CPM 10 whether the ESM 8 is run in open loop mode (OLM) 15 or closed loop mode (CLM) 20. In OLM 15, the clinician 7 keys-in stimulation parameter values to the UIM 9. In CLM 20, the ESM 8 calculates the stimulation parameter values in an automatic mode without clinician 7 or other human intervention, based on the programmed algorithms and memory information of the ESM 8.

Additionally, in one particular embodiment, a selector button is also used to indicate to the CPM 10 which of several pre-programmed protocols or “Therapy Modalities” may be administered to the patient 102. Electrical stimulation Therapy Modalities include, but are not limited to, micro stimulation (i.e., stimulation whose magnitude is measured in micro units such as microamperes or microvolts) or other spectrum of frequencies to elicit a corresponding resonance in biological tissues (i.e., targeting cells and their particular resonance frequencies). Microcurrent stimulation with conventional stimulation may significantly enhance patient response: in the case of patients with UI, increased energy levels and the cell formation effect of microstimulation may augment the benefits of the neural pathway improvements inherent to IVES. Electrical stimulation Therapy Modalities also include, but are not limited to, alternating frequencies of the stimulation signals or other combinations of frequencies to elicit different and complimentary reactions from each of the components of the bladder system. In one particular embodiment of the invention, the Therapy Modalities selected by the ESM 8 include a combination of both IVES and at least one electrical stimulation therapy modality. In one particularly preferred embodiment, the electrical stimulation therapy modality selected for use in combination with IVES is microstimulation.

Additionally, electrical stimulation Therapy Modalities may include, but are not limited to, Paired Associative Stimulation (“PAS”), which is a combination of low-frequency median nerve stimulation combined with transcranial magnetic stimulation over the motor cortex. PAS may provide supplemental positive functional effects as well since it intensifies the effect of somatosensory afferent nerve functionality by enhanced stimulation of cortical circuits. Thus, in one particular embodiment of the invention, the Therapy Modalities selected by the ESM 8 include a combination of both IVES and PAS modalities.

The Therapy Modalities selected in the ESM 8 and/or UIM 9 configures the SOM 37 to generate the electrical levels specified in the Stimulation Parameters Output Group, and correspondingly activate one or more electrodes 70, sensors 90 or other sensors and electrodes 91 to deliver electrical stimulation to the patient's treatment areas and obtain sensed information. More particularly, in one embodiment, the SOM 37 is the component that generates the output electrical stimulation therapy pulses based on commands by the CPM. The SOM 37 generates electrical stimulation pulses within a range of voltage, current, pulsewidth, frequency, waveform (shape, phase, amplitude), power, and total energy. In one embodiment, a waveform is a square pulse. Alternately, waveform shapes can be sawtooth, elliptical or another shape and still be in keeping with the present invention. Stimulation therapy pulses deliver total energy as a means to balance specific patient conditions. In one embodiment, the stimulation therapy pulse waveform phase is bi-phasic. However, in an alternate embodiment, a monophasic stimulation therapy waveform phase is provided by the SOM 37.

In one embodiment, the SOM 37 conveys the Stimulation Parameters Output Group corresponding to a Therapy Modality to the respective electrodes 70 or sensors 90 of the catheter 68, via the ESM connector 42 and catheter connector 43, or to the other sensors and electrodes 91. In one embodiment, the Therapy Modalities include, but are not limited to, a selection of one or more electrical stimulation signals based on one or more sensory input signals. More particularly, the Therapy Modalities described herein include, but are not limited to, a combination of IVES with other forms of electrical stimulation, based on one or more sensor inputs, which are driven or received on any of the electrodes 70, sensors 90 or other sensors and electrodes 91 (“Therapy Modality” or collectively, “Therapy Modalities”).

Table 3, below, summarizes one particular exemplary set of Therapy Modalities that includes electrical stimulation options include OLM or CLM, IVES with VES and one surface electrical stimulation (SES), and sensory inputs including one, two or three sensor inputs.

TABLE 3

| | EXAMPLE | EXAMPLE | EXAMPLE |
|---|---|---|---|
| | GROUP ONE | GROUP TWO | GROUP THREE |
| | (28 permutations) | (28 permutations) | (84 permutations) |
| ELECTRICAL | One of: | One of: | One of: |
| STIMU- | a) OLM IVES or | a) OLM IVES or | a) OLM or |
| LATION | b) CLM IVES. | b) CLM IVES; | b) CLM IVES; |
| OPTIONS: | | Plus VES. | Plus VES; |
| | | | Plus one of: |
| | | | a) pelvic floor |
| | | | SES, |
| | | | b) urethral SES or |
| | | | c) rectal SES. |
| SENSOR | One, two or three | One, two or three | One, two or three |
| INPUT | of: | of: | of: |
| OPTIONS: | a) EMG of pelvic, | a) EMG of pelvic, | a) EMG of pelvic, |
| | b) Intravesical | b) Intravesical | b) Intravesical |
| | Pressure, | Pressure, | Pressure, |
| | c) Bladder | c) Bladder | c) Bladder |
| | Impedance or | Impedance or | Impedance or |
| | d) IntraUrethral | d) IntraUrethral | d) IntraUrethral |
| | Pressure. | Pressure. | Pressure. |

Table 3 illustrates various embodiments of groups of permutations based on an example of sensor inputs available for use with OLM IVES or CLM IVES, with SES and/or VES. In alternate embodiments, additional electrical stimulation electrodes could be used including, but not limited to, the perineum, pelvic floor area, urethral area, rectal area, a specific muscle or other surface or musculature surface areas or implanted electrodes. In further alternate embodiments, additional sensors could be used including, but not limited to, surface sensors, implanted sensors or special probes positioned over specific locations on the body including, but not limited to, direct and indirect measurement of electrical, mechanical and chemical activity related to the bladder and its control, such as the perineum, pelvic floor, urethral area, rectal area, a specific muscle, other areas within the urinary system, transcranial magnetic stimulation sensors positioned over the motor cortex, or other sensors providing EEG, EMG, ultrasonic, pressure, biological or other measured parameters.

In one particular embodiment, the clinician 7 configures the UIM 9 to calculate Stimulation Parameter values in constant-current or constant-voltage mode. Then, if the ESM 8 is set to run in OLM 15, the clinician 7 keys-in the initial conditions for the desired Stimulation Parameter values, including, but not limited to, settings for voltage, current, pulsewidth, frequency, waveform shape, waveform phase, waveform amplitude, total power, and total energy.

If the ESM 8 is set to run in CLM 20, the UIM 9 downloads updated heuristic information and other information, if any, from the RSM 101 by a data connection such as the internet or other wired or wireless connectivity scheme. The UIM 9 then supplements existing information in to MM 11 by storing the downloaded information also into the MM 11, making it available to the CPM 10 for further processing. The CPM 10 reads the information out of the MM 11 and executes algorithms in the AM 12 (which may be a processor, microcontroller, etc.) to calculate settings for the present-state Stimulation Parameters as a function of the information read out of the MM 11. The information stored into and read out of the MM 11 include, but are not limited to, Measured Parameters collected by the CVMM 38, Sensor Parameters collected by the CVMM 38, Calculated Parameters collected by the AM 12, OLM inputs and Feedback Response Parameters collected by the UIM 9 or RFM 100, Clinical Information collected by the RSM 101, Aggregated Parameter Models collected by the RSM 101 and Therapy Optimization collected by the RSM 101, and other centrally-stored information that the CPM 10 uses for parameter calculations (collectively, the “Stimulation Parameter Inputs”).

In an embodiment, if the ESM 8 is set to run in CLM 20 and algorithms executed in the AM 12 determine that the Therapy Modality should be adjusted (e.g., in addition to IVES, requiring other external sensors (i.e., of other sensors and electrodes 91) to be placed on the perineum, urethral sphincter, bladder wall, bladder neck, or other bladder-system areas), then the AM 12 halts the algorithm. The UIM 9 displays an appropriate message on the display so that a clinician 7 or patient 102 may place the other external sensors 91 as needed. The ESM 8 waits until the clinician 7 or patient 102 signals the ESM 8 to resume operation by keying in the appropriate command into the UIM 9, then continues running in CLM 20.

Central Processor Module 10

In one particular embodiment, the CPM 10 is a programmable central processor module, such as a microprocessor or an embedded control processor. The CPM 10 selects and receives input data from several sources. The CPM 10 obtains input data by the patient 102 or clinician 7 via the UIM 9. The CPM 10 obtains inputs including, but not limited to, Clinical Information, Feedback Response Parameters and Aggregated Parameter Models from the RSM 101 via the UIM 9 as well as from the RSM 101 via the MM 11. The CPM 10 obtains inputs including, but not limited to, Stimulation Parameters, Measured Parameters and Sensor Parameters from the FIM 39. The CPM 10 retrieves (and subsequently stores) various data to and from MM 11, including, but not limited to, Feedback Response Parameters, the Baseline Stimulation Parameters, Aggregated Parameter Models, and other Clinical Information. Based on these inputs, the CPM 10 executes appropriate algorithms in the AM 12 to determine appropriate Stimulation Parameter values, and controls the SOM 37 to generate and convey the corresponding electrical Stimulation Parameters Output Group to the patient 102.

Memory Module 11

In an embodiment, the MM 11 is a fixed, persistent read/write computer memory storage module. The CPM 10 retrieves from and stores into the MM 11 various information, including, but not limited to, transitory data, semi-permanent and permanent data. Transitory data includes, but is not limited to, temporarily stored information used when the AM 12 calculates Stimulation Parameters or performs other interim calculations. Semi-permanent and permanent data include, but is not limited to, data referenced when executing calculations (such as Clinical Information) or stored after the conclusion of the Therapy Modality for later reference (such as Stimulation Parameters, Measured Parameters and Feedback Response Parameters), typically pertaining to one or more patient's treatment history. Permanent data also include, but is not limited to, constant values, such as Safety Tolerances (defined below).

Algorithmic Module 12

In an embodiment, the AM 12 comprises an algorithmic logic unit or computational engine and accompanying logic and circuits, which executes algorithms and logic functions. The algorithms or logic functions comprise code stored in read-only memory locations, firmware stored in nonvolatile or semi-permanent memory locations, or software stored in the MM 11.

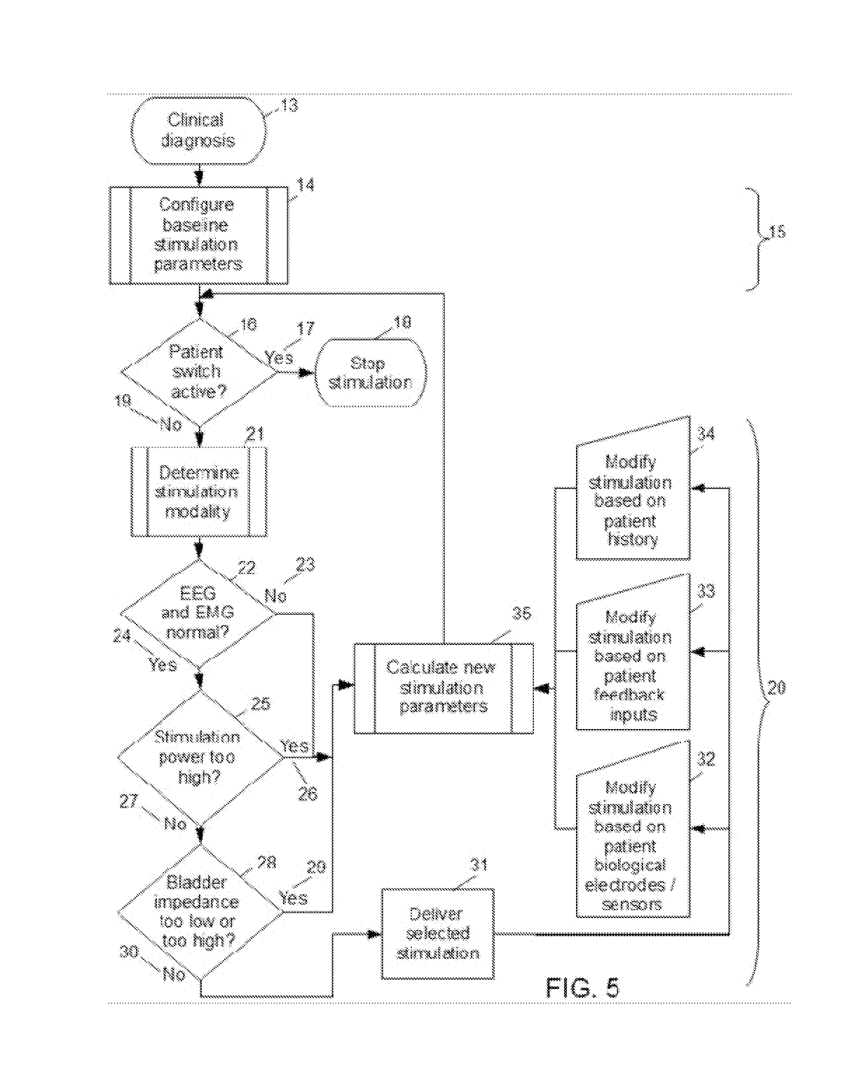

Referring now to FIG. 5, there is shown a method in accordance with one particular embodiment of the present invention. In one embodiment, the method of FIG. 5 is stored in non-transitory memory and executed by the AM 12 as directed by the CPM 10 within the ESM 8. First, a clinical diagnosis is made by the clinician 7 to determine the type and characteristics of a patient's 102 UI condition. Step 13. The clinician 7 then decides upon a prescriptive treatment based on the diagnosis, and enters into the UIM 9 a code corresponding to the diagnosis, or specific parameters and settings for the initial values of the Stimulation Parameters (“Baseline Stimulation Parameters”), which the CPM 10 stores in the MM 11 for subsequent processing. Step 14.

In an embodiment, any Stimulation Parameter not keyed in by the clinician 7 is configured by the CPM 10 to the AM 12 by reading Baseline Stimulation Parameters from memory locations in the MM 11. Such memory locations are designated for storing default values for use as initial treatment settings, or values corresponding to a particular diagnosis code, if any, entered by the clinician 7. In an embodiment, such memory locations are configured with values corresponding to diagnoses, including, but not limited to:

Diagnosis: Hyper tonic detrusor. Baseline Stimulation Parameters comprise values to perform either one or the combination of the following functions:
Inhibit detrusor contractions;
Activate detrusor contractions;
Relax detrusor and increase bladder capacity;
Relax perineum.

Diagnosis: Hypo tonic detrusor. Baseline Stimulation Parameters comprise values to perform either one or the combination of the following functions:
Activate detrusor contractions;
Increase detrusor tone;
Relax perineum;

Diagnosis: Hypo tonic detrusor—Spastic perineum. Baseline Stimulation Parameters comprise values to perform either one or a combination of the following functions:
Activate detrusor contractions;
Relax perineum.

Diagnosis: Detrusor tone too high and erratic. Baseline Stimulation Parameters comprise values to perform either one or a combination of the following functions:
Decrease the number of disorganized contractions;
Reduce detrusor tone;
Relax perineum;

Alternative embodiments include, but are not limited to, additional sets of treatment parameters corresponding to additional diagnoses, which supplement the list of stored Baseline Stimulation Parameters. Such additional sets of Baseline Stimulation Parameters may be updated into the MM 11 during the initial factory configuration, in the field as a memory update, or as part of normal unit operation. In normal unit operation, the clinician 7 manually enters new or updated values for Baseline Stimulation Parameters into the UIM 9, or downloads new or updated values for Baseline Stimulation Parameters from the RSM 101.

If the ESM 8 is configured to run in OLM 15, the AM 12 executes one iteration of a treatment cycle to the patient 102. If the ESM 8 is configured to run in CLM 20, after executing the first iteration of a treatment cycle to the patient 102, the AM 12 calculates subsequent next-state values for the Stimulation Parameters based on the algorithms described below, used during each subsequent iteration of a Therapy Modality session.

In an embodiment, values retrieved for the initial conditions of Stimulation Parameters from memory locations for two common sample diagnoses include, but are not limited to:

TABLE 4

| | | URGE IN- | STRESS IN- |
|---|---|---|---|
| | | CONTINENCE/ | CONTINENCE/ |
| | | HYPERTONIC | HYPOTONIC |
| PARAMETER | UNITS | DETRUSOR | DETRUSOR |
| Voltage | volts | 20 | 20 |
| Current | milliamps | 10 | 10 |
| Pulsewidth | microseconds | 350 | 350 |
| Frequency | Hz | 5 | 50 |
| Waveform shape | | Square | Square |
| Waveform phase | | Biphasic | Biphasic |
| Power | | Various levels | Various levels |
| Energy | | Various levels | Various leves |
| Stimulation | | IVES | IVES |
| Target Area: | | | |

Additionally, the AM 12 checks the status of the PSS 40. Step 16. If the signal is enabled (step 17), the AM 12 signals a stop-exit condition and the CPM 10 stops execution of the treatment. Step 18. If the PSS 40 signal is not enabled (step 19), the AM 12 continues to the next step.

In the present embodiment, the AM 12 determines the Therapy Modality. Step 21. In OLM 15, the Therapy Modality is configured based on settings keyed into the UIM 9 when the clinician 7 initializes the desired modality. In CLM 20, the Therapy Modality is configured when the CPM 10 reads the appropriate values from memory storage locations in the MM 11 designated for storing the Baseline Stimulation Parameter values (if the CPM 10 is executing its initial cycle) or the calculated next-state Stimulation Parameter values (if the CPM 10 is executing a subsequent CLM 20 cycle).

In an alternative embodiment, in CLM 20, when the AM 12 configures the Therapy Modality it also displays a status summary on the UIM 9 interface screen. If any changes to the Therapy Modality are prescribed, the CPM 10 stops execution of the algorithm, waits for the clinician 7 or patient 102 to make any appropriate physical changes (e.g., to re-position the catheter 68 or place any external Other Sensors and Electrodes 91, as described in the Method of Use section below), and the CPM 10 resumes execution of the algorithm after the clinician 7 or patient 102 keys the UIM 9 to resume.

In step 22, the AM 12 checks the measurement levels for EEG and EMG parameters or other Sensor Inputs measured and presented by the respective sensors 91 to ensure the EEG and EMG parameters are within normal levels. If the EEG and EMG parameters or other Sensor Parameters are not within normal levels (step 23), the AM 12 recalculates the Stimulation Parameters after making an adjustment to the power levels. Step 35. If the EEG and EMG parameters or other Sensor Parameters are within normal levels (step 24), the AM 12 continues to the next step.

In step 25, the AM 12 executes a comparison algorithm to check whether the Stimulation Parameters of power, voltage and current values corresponding to the configured Therapy Modality are within pre-defined safety tolerance levels (“Safety Tolerances”), where the value of power may result in tissue burns if administered to the patient 102. If the Stimulation Parameter values are greater than the Safety Tolerances (step 26), the AM 12 recalculates the Stimulation Parameters after making an adjustment to the power levels, as discussed below. Step 35. If the Stimulation Parameters are within the Safety Tolerances (step 27), the AM 12 continues to the next step.

In step 28, the AM 12 executes a comparison algorithm to check whether the Measured Parameter values and Calculated Parameters (defined below) correspond to a low-impedance situation in the bladder system that falls outside the Safety Tolerances, where excessive current may result in tissue burns if administered to the patient 102. If the Measured Parameters or Calculated Parameters fall outside the Safety Tolerances (step 29), the AM 12 recalculates the Stimulation Parameters after making an adjustment to the voltage or current levels, as discussed below. Step 35. If the Measured Parameters and Calculated Parameters are within the Safety Tolerances (step 30), the AM 12 continues to the next step.

Based on the settings configured for the Stimulation Parameters, the CPM 10 enables the conveyance and delivery of the Therapy Modality treatment cycle as the Stimulation Parameters Output Group to the patient 102. Step 31. The CPM 10 also stores the present-state conditions for future analysis. In an embodiment, the CPM 10 stores the Stimulation Parameter Inputs into the MM 11. The CPM 10 uploads the Stimulation Parameter Inputs via the UIM 9 to the RSM 101, as configured via a network, Internet or other wired or wireless connectivity scheme for further analysis and aggregation by the clinician 7.

After delivery of the selected stimulation (step 31), the AM 12 receives inputs in preparation for determining the next-state value of the Therapy Modality treatment cycle, i.e., using retro feedback gathered relative to the delivery of the selected stimulation. For example, in one embodiment, the AM 12 receives results conveyed by the FIM (39 of FIG. 4), including, but not limited to, the Stimulation Parameters, Measured Parameters, Sensor Parameters, and Calculated Parameters resulting from feedback and measurements from the patient 102 after administering the Therapy Modality. In an interim step, the AM 12 reads previous-state values of Stimulation Parameter Inputs from designated memory locations in the MM 11 to determine calculated values (the “Calculated Parameters”).

Note that, in the first iteration of CLM 20, previous-state values of Stimulation Parameters Inputs do not yet exist; therefore, in the first iteration of CLM 20 the AM 12 uses an aggregated value for the Stimulation Parameter Inputs, as stored in designated memory locations in the MM 11. In subsequent iterations of CLM 20, the AM 12 uses the patient's 102 own treatment results (as distinguished with a Patient ID, defined below) as captured in the previous-state Stimulation Parameter Inputs and stored in designated memory locations in the MM 11. Step 32.

Calculated Parameters include, but are not limited to:

“Bladder System Impedance,” defined as the Measured Parameter voltage (“Bladder System Voltage”) divided by Measured Parameter current (“Bladder System Current”);

“Bladder Compliance,” defined as the Sensors Parameter bladder volume divided by Sensors Parameter bladder internal detrusor pressure;

“Total Charge,” defined as the integration of the amount of Bladder System Current over the total duration of stimulation treatment;

“Total Power,” defined as the square of Bladder System Current times the Bladder System Impedance, or the square of Bladder System Voltage divided by the Bladder System Impedance;

“Total Energy,” defined as the integration of Total Power over the total duration of stimulation treatment; and

“Statistical Measures,” defined as calculations performed by comparing each present-state value of the Stimulation Parameter Input to the previous-state value for each Stimulation Parameter Input retrieved from its designated memory location in the MM 11. Calculated comparisons include, but are not limited to, a delta offset, trend value (weighted moving average), mean, standard deviation, variance, maximum, minimum, median, and other statistical measurements, and storing the results into designated memory locations in the MM 11.

Additionally, the AM 12 receives and processes data conveyed by the UIM 9, the MM 11 or the RSM 101 including, but not limited to, OLM 15 inputs (as discussed above) or conveyed by the patient 102 via the RFM 100 including, but not limited to, Feedback Response Parameters (if any). Step 33.

Further, in the present particular embodiment, the AM 12 receives and processes Clinical Information and Aggregated Parameter Models and Therapy Optimization, which are representative of the patient's 102 treatment history, entered by a clinician 7 and stored in the RSM 101. Step 34.

In one particular embodiment, the AM 12 determines the magnitude of adjusting the Stimulation Parameter values. Step 35. The AM 12 either: (i) recalculates values as a result of a safety; or (ii) determines the next-state value of each Stimulation Parameter as a function of present-state values of Stimulation Parameter Inputs when the CPM 10 is configured to run in CLM 20.

In an embodiment, an example range for the Stimulation Parameters includes, but is not limited to, the ranges below:

| | | |
|---|---|---|
| “Stimulation Voltage”: | 0-120 | volts; |
| “Threshold Two:” | <30 | volts; |
| “Microstimulation Current (range one)”: | 0-1,000 | micro amps; |
| “Microstimulation Current (range two)”: | 0-2,000 | micro amps; |
| “Stimulation Current (range three)”: | 0-100 | mA; |
| “Threshold One”: | <70 | mA; |
| “Stimulation Pulsewidth”: | 0-1,400 | microseconds; |
| “Stimulation Frequency”: | 0-500 | hz; |

| | |
|---|---|
| “Stimulation Waveform Shape”: | Square, triangular sawtooth, |
| sinusoidal; | |
| “Stimulation Waveform Phase”: | Monophasic, biphasic, |
| interferential | |

TENS asymmetrical modulated, bursts, microcurrent;

| | | |
|---|---|---|
| “Stimulation Energy”: | <10 | Watts/cm2; |

25 mW/cm2 - relative to patient safety;

| | |
|---|---|
| “Total Stimulation Power”: | Threshold one: <4.2 mA/cm2; |
| | Threshold two: <10.0 ma/cm2; |

“Stimulation Target Areas: Any nerve, tissue, fiber or group of cells that directly or indirectly influences the Urinary System and its surroundings including, but not limited to: IVES, VES, urethral area, perineum area, specified muscle area, other specified area within the urinary system, transcranial nerve region over the motor cortex, other SES or implanted electrode areas.

(i) In one embodiment, when a safety condition is triggered the AM 12 recalculates the Stimulation Parameters after making various adjustments 35. In an embodiment, the AM 12 first adjusts the power level by reducing the voltage level (if constant-current is specified in the Therapy Modality) or reducing the current level (if constant-voltage is specified in the Therapy Modality) by a fraction. After making the adjustment, the AM 12 recalculates all the Stimulation Parameter values. The AM 12 then re-iterates through the algorithm beginning from the third step described above 16. In one particular embodiment, the fraction is 1%. It is contemplated that other fractions may be used to reduce the values.

An alternative embodiment of the algorithm may also reduce the power level by reducing the pulsewidth or the frequency; however, reducing pulsewidth is preferred over reducing frequency. In one embodiment, the AM 12 calculates the maximum pulsewidth as ⅛*1/frequency. Alternative embodiments include, but are not limited to, other higher or lower limits.

An alternative embodiment of the algorithm makes an adjustment to the Stimulation Parameters to clamp the values at pre-programmed maximum values, thereby limiting the maximum electrical stimulation energy that may be administered to the patient 102 and avoiding a condition that may lead to exceeding the Safety Tolerance limits. In another alternative embodiment, power may be reduced by reducing the pulsewidth by a fraction. In an embodiment, the fraction is 1%. It is contemplated that other fractions may be used to reduce the frequency values.

(ii) In an embodiment, in CLM 20, the AM 12 calculates next-state values for the Stimulation Parameters. In an embodiment, when determining the next-state value of each Stimulation Parameter as a function of present-state values of Stimulation Parameter Inputs (including, but not limited to, Statistical Measures), the CPM 10 adjusts the Stimulation Parameters independent from and without need for intervention by the clinician 102. The CPM 10 executes algorithms in the AM 12 to determine the next-state values for the Stimulation Parameters 35 as a function of the Stimulation Parameter Inputs.

In an embodiment, when the UIM 9 is set to constant-current mode, an algorithm in the AM 12 keeps the Stimulation Current constant and adjusts the Stimulation Voltage as a function of the present-state value of Bladder System Impedance and its variation relative to the Statistical Measures calculated for the Bladder System Impedance and other relevant Stimulation Parameter Inputs. When the UIM 9 is set to constant-voltage mode, an algorithm in the AM 12 keeps the Stimulation Voltage constant and adjusts the Stimulation Current as a function of the present-state value of Bladder System Impedance and its variation relative to Statistical Measures calculated for the Bladder System Impedance and other relevant Stimulation Parameter Inputs.

In an embodiment, the AM 12 adjusts the Stimulation Parameter as a function of the magnitude of variation comprising the present-state value of any Stimulation Parameter Input compared to its previous-state and Statistical Measures values (the “Adjustment Function”). In an embodiment, the Adjustment Function is the rate of change determined by comparing the present-state value with its previous-state value or a Statistical Measures value.

In an alternative embodiment, the Adjustment Function is a fractional value multiplied times the rate of change determined by comparing a present-state value compared to its previous-state value or a Statistical Measures. Fractional values may include, but are not limited to, 25%, 10%, 1%, or 0.1%, log or natural log.

In an embodiment, the selected Statistical Measure is the mean. In an alternative embodiment, the selected Statistical Measure is the median, or some other statistical measurement. The computation of previous values and analysis of Statistical Measures for the Stimulation Parameter Inputs provide information about the patient's 102 response, which enables the algorithmic computations for Stimulation Parameters.

In an alternative embodiment, the Adjustment Function is field-updateable by downloading Clinical Information from the RSM 101 into the UIM 9.

In an embodiment, the CPM 10 also determines the magnitude of adjusting the next-state value of each Stimulation Parameter as a weighted function. In an embodiment, the AM 12 applies weightings that prioritize the impact of each of the next-state Stimulation Parameter values. The weightings are chosen as a multiplicative integer beginning with an integer of “1” for the lowest assigned priority and subsequently incrementing the integer to correspond with higher assigned priorities. In an embodiment, priorities are assigned, in the order from lowest to highest priority, as:

Total Power=1;

Energy=2;

Waveform Shape=3;

Waveform Phase=4;

Pulsewidth=5;

Current=6;

Voltage=7; and

Frequency=8

(collectively, the “Weighted Priorities”). Alternatively, other priority scales may be utilized.

In an alternative embodiment, the AM 12 adjusts the Weighted Priorities as a function of applying a fixed fractional value, such as multiplying by 25%, 10%, 1%, or 0.1%, or another percentage, or by multiplying by an incremental factor that varies as a logarithmic or exponential function.

In one embodiment, when in CLM 20 the AM 12 executes iterations through the entire algorithm until a predefined maximum total treatment duration is reached. In an embodiment, the maximum total treatment duration is stored in the MM 11 in a designated memory storage location. In an alternative embodiment, the AM 12 executes iterations through the entire algorithm until a predefined success criteria is met, defined as an event occurring when a comparison of the present-state values for the Stimulation Parameter Inputs matches values retrieved from a memory storage location in the MM 11 designated for storing the success criteria values for the respective Stimulation Parameter Inputs.

Stimulation Output Module 37

Referring again to FIG. 4, in one embodiment, the SOM 37 is, or includes a digital-to-analog converter, an analog voltage and/or current parametric forcing unit, multiplexers, and accompanying logic and circuits. When the ESM 8 is in drive-mode (i.e., energizing and conveying the analog levels needed during treatment), the Stimulation Parameter values determined by the CPM 10 and the AM 12 are conveyed electrically to the SOM 37. The SOM 37 then converts the digital values to their equivalent analog signals for the respective Stimulation Parameters, driving them as the Stimulation Parameters Output Group to the appropriate electrodes 70, sensors 90 or other sensors and electrodes 91, and the CVMM 38. These signals include, but are not limited to, voltage, current, pulse width, frequency, and waveform (e.g., shape, phase and amplitude), and external signals directed to other sensors and electrodes 91 including, but not limited to, surface electrodes or implanted electrodes to deliver electrical stimulation to specific locations on the body including, but not limited to, the perineum, pelvic floor, urethral area, rectal area, a specific muscle or other areas within the urinary system. The SOM 37 conveys electrically the Stimulation Parameters Output Group via the ESM connector 42 and the catheter connector 43 to the respective electrode 70 and/or sensor 90 in the catheter 68 and/or other sensors and electrodes 91 as a function of the Therapy Modality selected in the UIM 9.

In an embodiment, the SOM 37 also directs stimulation to other nerves and central control points via Other Sensors and Electrodes 91 located at specific locations on the body including, but not limited to, electrical, mechanical and chemical functions related to the bladder and its control. In an embodiment, the Therapy Modalities utilize one or more of the electrodes 70, sensors 90, or other sensors and electrodes 91, as described below

Current/Voltage Measuring Module 38

The CVMM 38 is a component provided to ensure the integrity of the output electrical signals generated by the SOM 37 as well measure the responsive signals from the patient 102. This module 38 determines actual energy delivered to the patient 102, which is dependent on the signals generated by the SOM 37, as well as the patient's bladder system's voltage, current and other measurements. Referring back to FIG. 4, in one embodiment, the CVMM 38 includes voltage and current parametric measurement units (autoranging and capable handling low to high magnitudes of voltage or current), analog-to-digital converters, integrators, multiplexers, and accompanying logic and circuits, and any needed analog and digital circuit filters, digital signal processing circuits, and accompanying logic and circuits, for performing any needed signal filtering and rehabilitative processing due to the presence of noise or attenuation.

When the ESM 8 is in drive-mode, the CPM 10 connects the bus signals carrying the analog levels of the Stimulation Parameters Output Group (i.e., voltage, current, pulse width, frequency, and waveform shape, phase and amplitude) to the CVMM 38, so that the CVMM 38 measures the levels driven by the SOM 37. When the ESM 8 is in receive-mode, the CVMM 38 measures the analog levels conveyed into the CVMM 38 from the patient 102 via the Sensors 90 and their inherent electrical conductors, the catheter connector 43, and the ESM connector 42. The analog levels measured by the CVMM 38 include, but are not limited to, the effect of the “load” of the patient's 102 bladder system. Similar to drive-mode, the analog signals include, but are not limited to: voltage, current, pulse width, frequency, and waveform (e.g., shape, phase, amplitude); the CVMM converts the analog signals into digital representations of their values (collectively, the “Measured Parameters”).

The CVMM 38 also receives input signals conveyed electrically from the Other Sensors and Electrodes 91 via standard input connection ports. The input signals include, but are not limited to, signals representing: bladder system impedance, EEG signals, bladder-system EMG, vaginal pressure sensors, intra bladder detrusor pressure sensors, urethral sphincter pressure sensors, urethral sphincter closure pressure, intra bladder (or intravesical) pressure sensors, bladder-system EMG, bladder volume, residual urine volume after voiding, and other biological signals (i.e. signals correlative of bodily processes involved in the neurological and physiological function of the bladder system including, but not limited to, meridian voltage points, bladder mucosa, mechanoreceptors, somatic innervations, and the transcranial nerve region over the motor cortex or other areas); the CVMM 38 converts any analog signals into digital representations of their values (collectively, “Sensor Parameters”).

In an embodiment, the Sensor Parameters include, but are not limited to, values representing measurements comprising the following sensor inputs and ranges:

TABLE 5

| PARAMETER/SENSOR | VALUE | REMARKS |
|---|---|---|
| Bladder impedance | 20-2,000 Ohms | Calculated in the AM 12 |
| | | as a function of Bladder |
| | | System Voltage and |
| | | Bladder System Current |
| Bladder pressure | 0-15 mmHg | Provided by Other |
| | (normal) | Sensors and Electrodes |
| | 15-50 mm Hg | 91 |
| | (elevated) | |
| EMG signals from any | Various | Provided by Other |
| nerve in the bladder | | Sensors and Electrodes |
| system to the brain | | 91 |
| and vice versa | | |
| (efferent and | | |
| afferent) | | |
| Vaginal pressure | | Provided by Pther |
| (in cm H(2) O) | | Sensors and Electrodes |
| Cough | 40.0-133.7 | 91 |
| Standing | 15.0-28.5 | |
| Supine exercise | 6.0-91.9 | |
| Urethral pressure | 25-140 cm H(2) | Provided by Other |
| | O | Sensors and Electrodes |
| | | 91 |
| | | More provocative methods |
| | | of pressure measurement, |
| | | which simulate |
| | | physiological conditions of |
| | | the urethra, may provide |
| | | more information on |
| | | sphincter efficiency. |
| Biological | Various | Provided by Other |
| sensors (90) | | Sensors and Electrodes |
| | | 91 |
| | | Sensors the body has that |
| | | adjust function of the |
| | | bladder system, including, |
| | | but not limited to, meridian |
| | | voltage points, bladder |
| | | mucosa, |
| | | mechanoreceptors, |
| | | somatic innervations and |
| | | others. |
| Pelvic tissue | <25 | Provided by Other |
| voltage (mV) | | Sensors and Electrodes |
| | | 91 |
| Back spine | <25 | Provided by Other |
| tissue impedance | | Sensors and Electrodes |
| (mV) | | 91 |

Alternative embodiments include, but are not limited to, modifications to the ranges of Sensor Parameters, as indicated based on heuristic feedback and other information collected over time in the form of Stimulation Parameter Inputs.

In an alternative embodiment, the SOM 37 and CVMM 38 share the same electrical conductors connecting the ESM 8 to the patient 102 via the ESM connector 42, the catheter connector 43, catheter Electrodes 70 and Sensors 90 and Other Sensors and Electrodes 91 and their inherent electrical conductors. In this case, the SOM 37 and CVMM 38 multiplex channels so that only one module at a time takes control of the signal path. When the ESM 8 is in drive-mode, the SOM 37 takes priority to drive its signals (i.e., the Stimulation Parameters Output Group). When the ESM 8 is in receive-mode, the CVMM 38 takes priority to receive its signals (i.e., the Measured Parameters).

The digital values representing the analog signals measured by the CVMM 38 are conveyed electrically by the CVMM 38 to the FIM 39 using internal electrical bus connections.

Patient Stop Switch 40

Referring again to FIG. 4, in an embodiment, the PSS 40 comprises an emergency-off or “kill switch” function, which enables and conveys an emergency-stop signal to the ESM 8 if the patient 102 presses the emergency shut-off switch. The PSS 40 conveys electrically the emergency-stop signal to the FIM 39 using standard input connection ports.

Feedback Input Module 39

The FIM 39 is a component to select the appropriate input values and route them for conditioning and use by the CPM 10. The inputs to the FIM 39 include, but are not limited to, electrical parameters measured in the CVMM 38, a signal from the PSS 40, and input signals provided by the sensors 90 and electrodes 70 configured in the catheter 68 and connectors. In one embodiment, the FIM 39 includes, but is not limited to, multiplexers, switches, and accompanying logic and circuits. The FIM 39 receives input signals conveyed electrically by the CVMM 38 including, but not limited to, Measured Parameters and Sensor Parameters. The FIM 39 also receives any emergency-stop signal conveyed electrically by the PSS 40. The FIM 39 conveys electrically its output signals to the CPM10 using electrical bus connections.

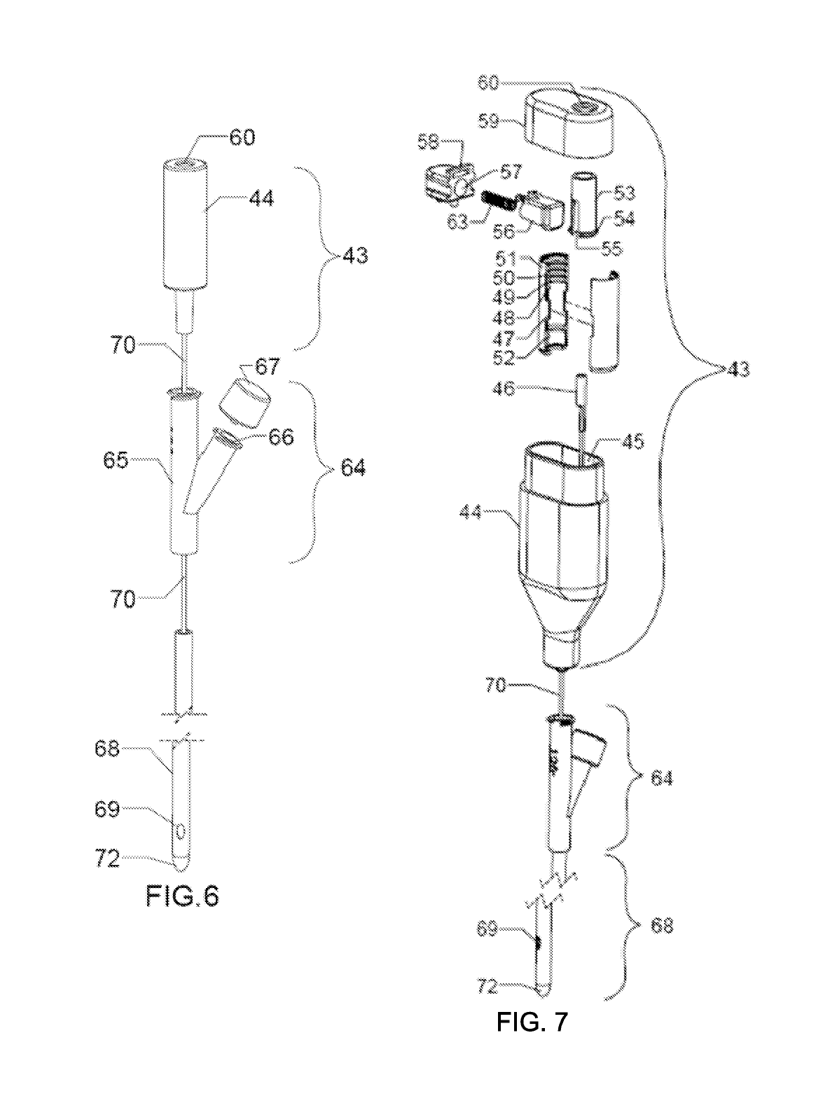

Referring now to FIG. 6, there is shown one particular embodiment of a catheter 68, useful with certain embodiments of the present invention. Catheter 68 and associated connectors may be a urinary catheter of standard outer dimension. Catheter 68 includes a catheter connector assembly 43 including a one-time connector housing 44. The catheter connector assembly 43 may include, but is not limited to, a mechanical and electrical connector that links the ESM (8 of FIG. 4) with the catheter connector 43 and any electrodes within the catheter 68. FIG. 6 also shows the housing cap channel pathway 60. In one particular embodiment, the housing 44 is formed of high-impact plastic or light-weight metal.

Catheter 68 includes an electrical conductor 70 passing through the axial lumen of the catheter connector assembly 43, the main shaft 65 of a Y-connector assembly or Y-connector 64, and the catheter 68. The Y-connector 64 permits a clinician (7 of FIG. 4) to introduce fluid into the lumen of the Y-connector 64 and the catheter 68 through a port 66 in the Y-connector 64. A Y-connector cap 67 is provided that would be removed when introducing the fluid. Catheter 68 of the present embodiment additionally includes an orifice 69 and a tip 72. Orifice 69 permits fluidic contact between the conductor 70 and intra-bladder fluid. The Y-connector 64 fluidly links the catheter with a fluid source to convey saline or other liquid through the lumen of the catheter, as may be appropriate, since the nature of IVES involves no specific contact area within the bladder.

One-Time Connector 43

Referring now to FIGS. 4 and 7, there is shown an exploded, elevational view of a one-time use catheter connector 43 in accordance with one embodiment of the present invention. The catheter connector 43 is connected to the ESM 8 (not shown) via the ESM connector 42, which is inserted into the catheter connector 43 through the housing cap channel pathway 60 positioned in the top of the catheter connector 43 and which makes electrical contact with an electrically conductive wire conductor 46. The catheter connector 43 is configured to permit the insertion of the ESM connector 42, but once ESM connector 42 is fully inserted into the catheter connector 43 and subsequently retracted, the catheter connector 43 blocks the insertion of any other ESM connector 42 into the catheter connector 43, hence the catheter connector 43 is “one-time use” only and intended to be disposed after use. In an embodiment, the catheter connector 43 is formed of high-impact plastic or light-weight metal.

In the embodiment of FIG. 7, the catheter connector 43 includes a connector housing cap 59 and a housing cap channel pathway 60. In an embodiment, the housing cap 59 is formed of high-impact plastic or light-weight metal, and provides a lid or cap for the top of the housing 44. As discussed above, the catheter connector 43 is configured to be used one-time only. Additionally, in one particular embodiment, the catheter 68 includes, but is not limited to, a catheter IVES and VES housing fabricated using an antibacterial coating to reduce infection. Both of these features facilitate usage of the system by the patient at home, as opposed to within the doctor's office or as an out-patient procedure.

The catheter connector 43 additionally includes a compressed spring 63. The spring 63 is configured to fit within a lock pin 56. The lock pin 56 is configured to fit within a lock pin chamber 57. The catheter connector 43 further includes a guide track 58 that is configured to guide the movement of the lock pin 56. In an embodiment, the spring 63 is formed of high-tensile strength, light-weight metal, and the lock pin chamber 57 is formed of high-impact plastic or light-weight metal.

The catheter connector 43 of the present embodiment also includes a barrier pin 53, a locking stub 54 and a barrier pin slot 55. In an embodiment, the barrier pin 53 is formed of high-tensile strength, high-impact plastic or light-weight metal. In the embodiment of FIG. 7 includes a barrier pin locking mechanism or barrier pin lock 47, and various locking positions of the barrier pin lock 47, including a barrier pin lock first unlocked ridge position 48, a second unlocked ridge position 49, a third unlocked ridge position 50, a fourth unlocked ridge position 51, and a locked ridge position 52. In an embodiment, the barrier pin lock 47 is formed of high-impact plastic or light-weight metal.

The embodiment illustrated in FIG. 7 additionally includes a housing 44, a lumen or housing channel lumen 45 passing within, and extending in the direction parallel to, the central axis of the housing 44, and a female plug and electrically conductive wire conductor (or “conductive plug”) 46. In an embodiment, the housing 44 is formed of high-impact plastic or light-weight metal. Although it is described that the catheter connector 43 utilizes a female connector to terminate the conductive plug 46, this is not meant to be limiting, as the connector 43 may alternatively utilize a male plug, if desired. The catheter connector 43 of the present embodiment can be fitted to a catheter assembly including a Y-connector 64 and an electrode 70 running through the lumen of the Y-connector 64, as shown. Additionally, the catheter 68 may include an orifice 69 and tip 72, as discussed in connection with the catheter 68 of FIG. 6.

In an embodiment, the housing channel lumen 45 includes a vertically formed pathway of diameter sized large enough to receive the insertion of the ESM connector 42 from the top, the horizontally formed and transversely mounted lock pin chamber 57 and its lock pin 56 and spring 63, the barrier pin 53 and barrier pin lock 47, and the conductive plug 46. The barrier pin 53, barrier pin lock 47, and conductive plug 46 are described in greater detail in the figures and paragraphs that follow.

In an embodiment, the lock pin chamber 57 comprises a cavity horizontally formed within the housing 44 and extending in the direction tranverse to and intersecting with the housing channel lumen 45. The lock pin chamber 57 is shaped to conform to and receive the dimensions of the lock pin 56 and house the lock pin 56 and the spring 63 mounted within and behind the lock pin 56. The lock pin chamber 57 defines the distance within which the lock pin 56 may move, under the expansion force of the spring 63. The expansion force of the spring tends to push the lock pin 56 out of the lock pin chamber 57 in the horizontal direction, which is transverse to the direction of the hollow axial path of the housing channel lumen 45. Therefore, as defined by the lock pin chamber 57, the lock pin 56 may move from a position against the outer wall of the housing cap 59 (i.e., fully retracted within the lock pin chamber 57) in the direction transverse and toward the axial path of the housing channel lumen 45 (i.e., fully extended out of the lock pin chamber 57). When in the position fully extended out of the lock pin chamber 57, the lock pin 56 intersects with, and blocks, the hollow axial path of the housing channel lumen 45.

Barrier Pin Lock 47

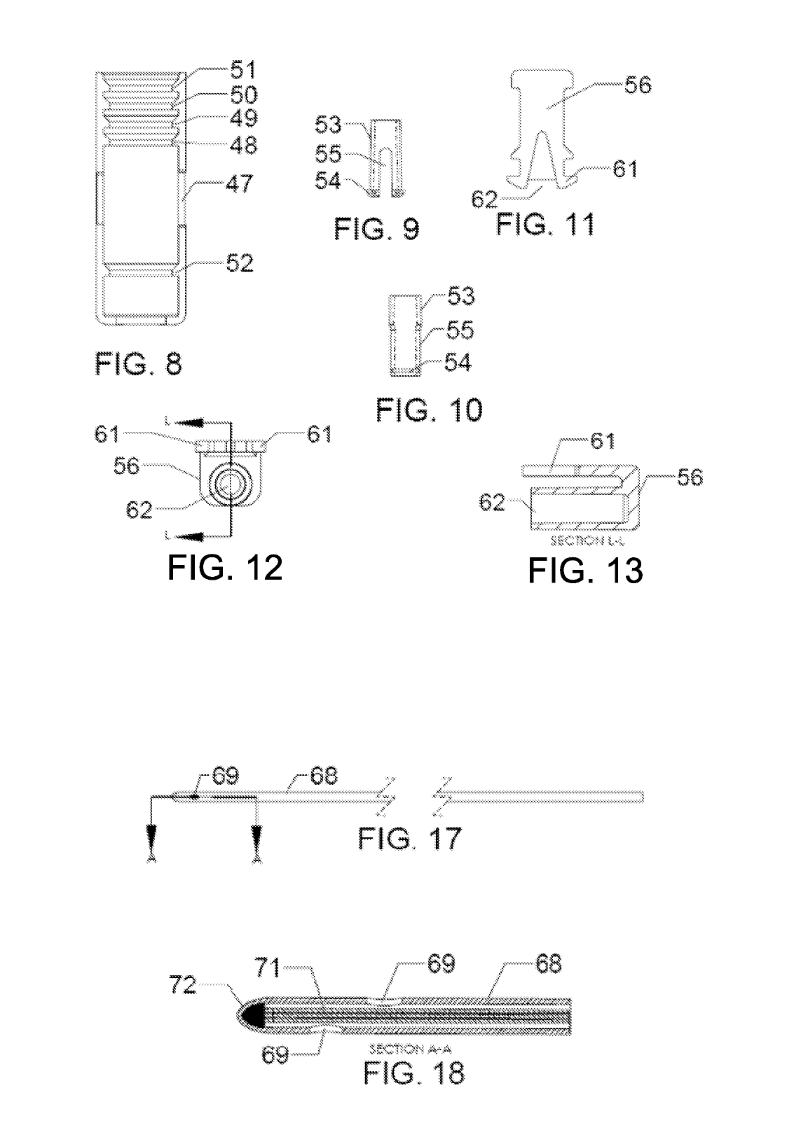

Referring now to FIGS. 7-13, the barrier pin lock 47 includes locking ridges 48 and 52. If desired, redundant locking ridges 49, 50, and 51 may, optionally, also be provided. The barrier pin lock 47 comprises a hollow cylinder formed of high-impact plastic or light-weight metal, axially aligned and positioned within the housing channel lumen 45. The outer diameter of the barrier pin lock 47 matches the inner diameter of the housing channel lumen 45, accounting for a manufacturing tolerance, so that the barrier pin lock 47 fits concentrically within the hollow axis of the housing channel lumen 45.

In an embodiment, the inner wall of the barrier pin lock 47 is configured with a locking ridge 48 that extends around the inner surface of the barrier pin lock 47, which acts with a ratcheting function upon the locking stubs 54 of the barrier pin 53. In an alternative embodiment, additional locking ridges 49 through 51 are configured within the inner wall of the barrier pin lock 47 to provide redundant stopping points against the locking stubs 54, to further stop the upward movement of the barrier pin 53 out of the barrier pin lock 47 if a clinician 7 or patient 102 attempts to extract the barrier pin 53 out of the barrier pin lock 47.

In an embodiment, the locking ridge 48 comprises a top surface forming a downward slanting direction with an obtuse angle (measured from the direction parallel to the inner vertical surface of the barrier pin lock 47 in the upward direction), and a bottom surface forming a substantially horizontal surface that is substantially orthogonal to the inner vertical surface of the barrier pin lock 47. In an embodiment, the top surface of the locking ridge 48 comprises a downward slanting direction that forms a ratcheting interface member of sufficient degree to provide sufficient lateral support strength to the ratcheting member. Certain embodiments of the present invention include, but are not limited to, a range for the obtuse angle from 115 degrees to 175 degrees.

The bottom surface forms a horizontal surface that is orthogonal to the inner vertical surface of the barrier pin lock 47 and provides substantial support lateral strength to the ratcheting support member of locking ridge 48. in an embodiment, each of the locking ridges 48 through 52 is formed similarly. In an alternative embodiment, the bottom surface of each of the locking ridges 48 through 52 forms a surface that slants downward or upward by a minimal degree from horizontal, for example within +− fifteen degrees from the horizontal.

Barrier Pin 53

FIG. 9 shows one embodiment of the present invention that includes, but is not limited to, a sideward looking elevation view of a barrier pin 53. This figure shows the barrier pin stubs 54 at the bottom of the barrier pin 53. This figure also shows an embodiment of two stress relief slots (“barrier pin slots”) 55 configured in the front and back surfaces of the barrier pin 53. In an embodiment, the barrier pin 53 includes a hollow cylinder formed of high-impact plastic or light-weight metal. The inner diameter of the barrier pin lock 47 matches the outer diameter of the barrier pin 53, accounting for a manufacturing tolerance so that the barrier pin 53 fits concentrically within the hollow axis of the barrier pin lock 47, and the barrier pin 53 may slide vertically up or down along the hollow axis of the barrier pin lock 47.

In an embodiment, at the bottom of each side of the barrier pin 53 is a lip or stub that projects in the outward radial direction (“locking stub”) 54. The locking stub 54 is formed of high-impact plastic or light-weight metal and has a tensile strength so that the locking stub 54 returns to its original position after being deflected or compressed. The locking stub 54 is configured with a ridge on the upper surface of the locking stub 54 to make contact and lock against any of the locking ridges 48-52 of the barrier pin lock 47, when the locking stub 54 moves past one of the locking ridges 48 through 52 in the downward direction. In an embodiment, the width of the locking stub 54 substantially overlaps the horizontal surface area of the ratcheting interface member of each of the locking ridges 48-52 sufficient to give mechanical stability and strength to stop the movement of locking stub 54 against one of the locking ridges 48-52 in the upward and outward direction. Certain embodiments of the present invention include, but are not limited to, a range for amount of overlap from 50% to 100%.

In one particular embodiment, the width of the barrier pin slot 55 is sufficient to give flexibility and stress relieve in the locking stub 54, as it pushes past the ratcheting interface member of one of the locking ridges 48-52 in the downward and inward direction. Certain embodiments of the present invention include, but are not limited to, a range for the width of the barrier pin slot 55 from a slit-cut to 80% of the diameter of the locking stub 54. In an alternative embodiment, the width of the locking stub 54 is a width substantially sufficient to move unrestrictedly past the locking ridges 48-52 when the barrier pin 53 moves within the barrier pin lock 47 in the downward direction, but catch and stop against any one of the locking ridges 48 through 52 when the barrier pin 53 moves in the upward direction and makes contact with one of the respective locking ridges 48 through 52.

In an embodiment, a slot or barrier pin slot 55 is formed in the front and back sides of the barrier pin 53 to relieve tension in the barrier pin 53 when the locking stubs 54 are compressed. The barrier pin slots 55 permit compression of the locking stubs 54 and facilitate the return of the locking stubs 54 to their original shape. The compression of the locking stubs 54 permit the barrier pin 53 to move past any of the locking ridges 48-52, while the expansion of the locking stubs 54 to their original shape cause the locking stubs 54 to make contact with, and lock against, one of the respective locking ridges 48-52 when the barrier pin 53 moves in the upward direction.

FIG. 10 shows certain embodiments of the present invention that include, but are not limited to, a sideward looking (rotated 90 degrees from FIG. 9) elevation view of the barrier pin 53. This figure shows the barrier pin stubs 54 at the bottom of the barrier pin 53. This figure also shows the slight cutout of the barrier pin slots 55 from the surface of the barrier pin 53. Referring back to FIGS. 7-13, each of the locking ridges 48-52 of the barrier pin lock 47 therefore permits the downward movement (i.e., into the barrier pin lock 47) of the barrier pin 53 as the locking stub 54 moves unrestrictedly past the angled top surface of one of the locking ridges 48 through 52, but stops the upward movement (i.e., out of the barrier pin lock 47) of the barrier pin 53 when the locking stub 54 catches and stops against the horizontal bottom surface of the locking ridge 48.

In one particular embodiment of the invention, the initial, factory-set default position of the barrier pin 53 within the barrier pin lock 47 is with the locking stub 54 positioned at locking ridge 48, so that the top of the barrier pin lock 47 is substantially flush with the top of the barrier pin 51.

Lock Pin 56

FIG. 11 shows certain embodiments of the present invention that include, but are not limited to, a downward looking elevation view of a lock pin 56. Apparent in the figure is a lock pin vane 61 that is configured to follow the channel of the lock pin chamber 57 and align the lock pin's 56 movement within the lock pin chamber 57. Also apparent in FIG. 11 is the hollow circular chamber or spring chamber 62 for the compressive spring 63 of FIG. 7. The width of the lock pin 56 is sufficiently large to block and close off the housing cap channel pathway 60 and housing channel lumen 45 and block the insertion of an ESM connector 42 into the housing cap channel pathway 60 and housing channel lumen 45. In one particular embodiment, the lock pin 56 comprises a cylindrical plug of high-impact plastic or light-weight metal.

FIG. 12 shows a rearward looking elevation view of the lock pin 56 and the boring of the circular spring chamber 62, whose diameter is selected to fit a spring 63 of FIG. 7, selected from one common in the art, and the width of the lock pin vane 61, which is configured to match the width of the lock pin chamber guide track 58 formed in the lock pin chamber 57.

FIG. 13 shows certain embodiments of the present invention that include, but are not limited to, a cross sectional view of the lock pin 56 at Section L-L. This figure shows the boring of the circular Spring Chamber 62, whose diameter is selected to fit a spring 63 and the lock pin vane 61 that extends the length of the lock pin 56. Alternative embodiments of the shape of the lock pin 56 include, but are not limited to, a square or rectangular plug.

Unlocked Position—ESM Connector 42 May be Inserted

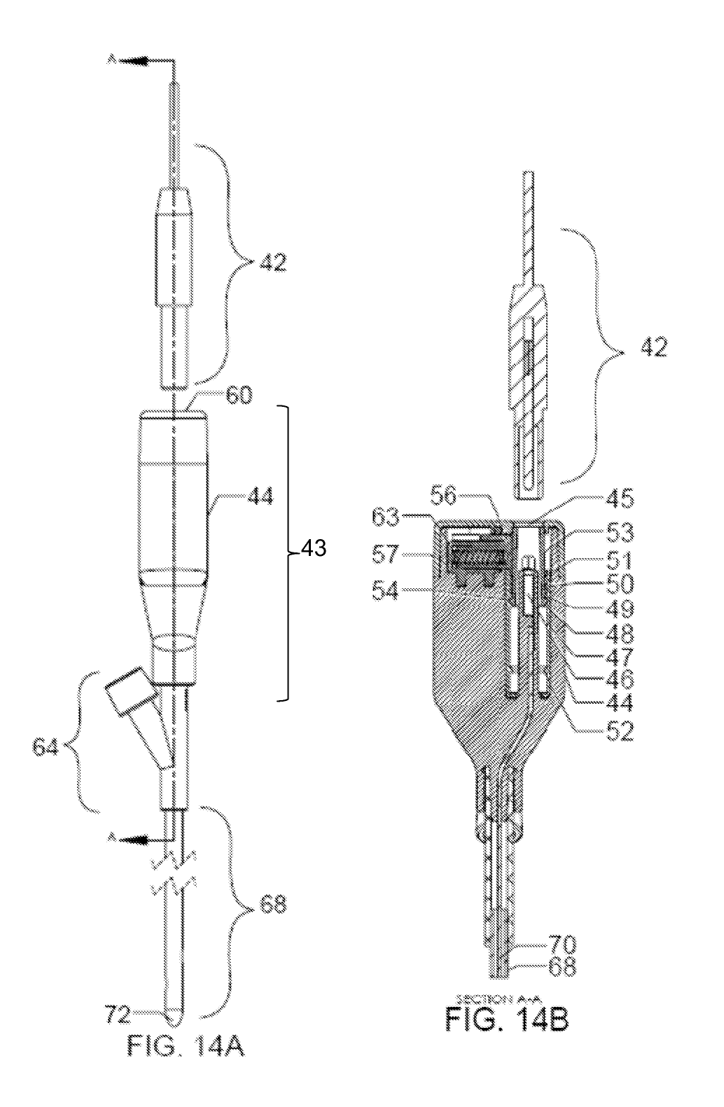

FIG. 14A illustrates certain embodiments of the present invention that include, but are not limited to, a side elevation view of an ESM connector 42, a one-time use connector 43 including a connector housing 44 and a catheter 68. For illustrative purposes, FIG. 14A shows the ESM connector 42 not inserted into the connector housing 44. The figure also shows a Y-connector 64.

FIG. 14B is a cross-sectional view taken at Section A-A of FIG. 14A of the ESM connector 42, a one-time use connector 43 comprising a connector housing 44, and a catheter 68. Among other things, FIG. 14B shows an electrode 70 connected to a conductive plug 46, which fits concentrically within the hollow axis of a barrier pin 53, which fits concentrically within the hollow axis of a locking pin lock 47. When the barrier pin 53 is in the upward position, the motion of the lock pin 56 is impeded from extending out of the lock pin chamber 57 across the housing channel lumen 45. Because the lock pin 56 does not block the housing channel lumen 45, the one-time use connector 43 is “unlocked,” and a clinician 7 or patient 102 (not shown) may freely insert the ESM connector 42 into the housing channel lumen 45.

In one particular embodiment of the invention, the initial, factory-set default position of the barrier pin 53 within the barrier pin lock 47 and housing channel lumen 45 is chosen so that the locking stubs 54 have a range of motion between locking ridge 51 and locking ridge 48 (encompassing interim positions at locking ridges 49 through locking ridge 51), which permits the barrier pin 53 to move up and down within the barrier pin lock 47 within that range of motion. Within that range of movement, the barrier pin 53 is positioned in front of the lock pin 56, thereby preventing the lock pin 56 from extending out of the lock pin chamber 57 under the force of the spring 63, and preventing the lock pin 56 from entering the housing channel lumen 45. Because the lock pin 56 does not enter the housing channel lumen 45, it does not block the housing channel lumen 45 and does not impede the insertion of an ESM connector into the housing channel lumen 45; hence, the catheter connector 43 is “unlocked.” FIG. 14B additionally, shows locking ridge 52, which is not engaged by the locking stubs 54 since the barrier pin 53 is in the upward and “unlocked” position.

Locked Position—ESM Connector 42 Inserted

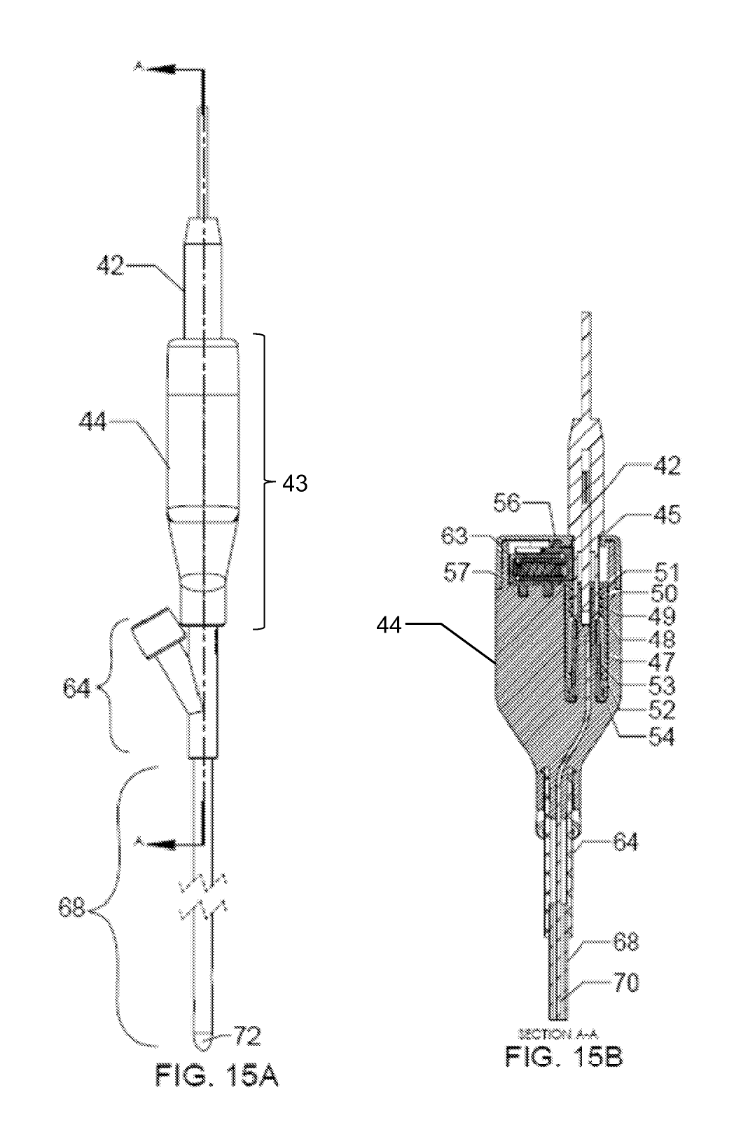

Referring now to FIGS. 15A and 15 B, there is shown one particular embodiment of the invention in which the catheter connector 44 is in a locked position. More particularly, an ESM connector 42, a one-time use connector 43 comprising a connector housing 44, and a catheter 68 having a Y-connector 64. For illustrative purposes, FIGS. 15A and 15B show the ESM connector 42 inserted into the connector housing 44.

FIG. 15B is a cross-sectional view taken at Section A-A of FIG. 15A showing the ESM connector 42, a one-time use connector 43 including a connector housing 44, and a catheter 68. As in FIG. 14B, FIG. 15B illustrates an electrode 70 connected to a conductive plug 46, which fits concentrically within the hollow axis of a barrier pin 53, which fits concentrically within the hollow axis of a locking pin lock 47. FIG. 15B shows an embodiment of the situation when the barrier pin 53 is in the downward position, after pushed downward by the insertion of the ESM connector 42. The sidewall of the ESM connector 42 impedes the motion of the lock pin 56 from extending out of the lock pin chamber 57 under the expansion force of the Spring 63, thereby preventing the lock pin 56 from entering the housing channel lumen 45.

As the clinician (7 of FIG. 4) or patient (102 of FIG. 4) pushes the ESM connector 42 into the housing channel lumen 45, in one embodiment, the female sleeve of the ESM connector 42 pushes the barrier pin 53 downward (i.e., into the barrier pin lock 47) and the locking stubs 54 pass-by each of the various locking ridges (i.e., each of locking ridges 51-48). As the locking stubs 54 pass-by the bottom locking ridge 52, the locking stubs 54 engage with, and become trapped at, locking ridge 52 when the locking stubs 54 move in the upward direction, so that the locking stubs 54 cannot be retracted past locking ridge 52 in the upward direction. Therefore, the barrier pin 53 is held and fully retracted within the barrier pin Lock 47, and prevented from any subsequent upward movement (i.e., out of the barrier pin Lock 47).

In one embodiment, while the ESM connector 42 is inserted into the housing channel lumen 45, the electrical connector inside the ESM connector 42 makes electrical connection with the male connector of the conductive plug 46. In one particular embodiment, the conductive plug 46, comprised of one or more electrically conductive wires, electrically connect with one or more electrode(s) 70, comprised of one or more electrically conductive wires within the catheter connector 43.

Locked Position—ESM Connector 42 Prevented from Insertion

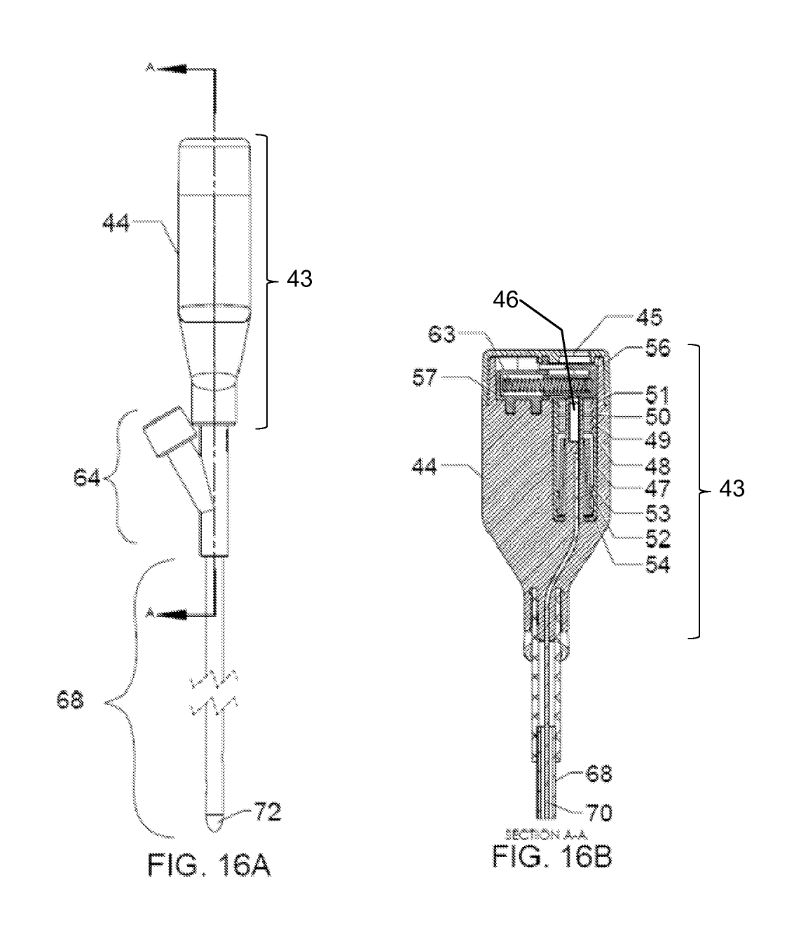

FIGS. 16A-16B show one particular embodiment of the invention of a one-time use connector 43 including a connector housing 44, engaged with a catheter 68 having a Y-connector 64. For illustrative purposes, the figure does not show an ESM connector 42 that could otherwise be inserted into the connector housing 44. FIG. 16B is a cross-sectional view taken at Section A-A of FIG. 16A of a one-time use connector 43 including a connector housing 44, and a catheter 68. As in FIG. 14B, the figure illustrates an electrode 70 connected to a conductive plug 46, which fits concentrically within the hollow axis of a barrier pin 53, and which fits concentrically within the hollow axis of a locking pin lock 47.

FIGS. 16A-16B show a locked position situation where the barrier pin 53 is in the downward position and the locking stubs 54 are aligned with, engaged with, and locked by the locking ridge 52. As in FIG. 15B, the locking stubs 54 pass-by each of the various locking ridges (i.e., each of locking ridges 51 through 48). In this downward, locked position, the barrier pin 53 does not prevent the lock pin 56 from entering the connector housing lumen 45; hence the expansion force of the spring 63 pushes the lock pin 56 out of the lock pin chamber 57 and into the vertical axial pathway of the connector housing lumen 45.

In the embodiment illustrated in FIGS. 16A and 16B, the lock pin 56 is pushed along the lock pin chamber guide track 58, sliding horizontally (i.e., in the direction transverse to the vertical axial direction of the housing channel lumen 45) and across the vertical axial pathway of the housing channel lumen 45. Because the position of the lock pin 56 blocks any further subsequent insertion of an ESM connector (42 of FIG. 14A-15B) into the housing channel lumen 45 of the one-time connector 43, the one-time connector 43 is “locked.”

Catheter Electrodes 70

The electrically conductive elements forming the electrodes 70 and sensors 90 and other sensors and electrodes 91 are, in the most preferred embodiment, comprised of electrically conducting and physiologically neutral conductors fabricated out of copper. As desired, the electrically conductive elements include, but are not limited to, silver, gold, platinum, stainless steel or other electrically conductive and physiologically inert metal or alloy.

Embodiments of IVES Electrodes 70

FIGS. 17 and 18 illustrate one particular embodiment of a catheter 68 for use with the present invention that includes an orifice 69 located at the proximal end (i.e., the end that would be most deeply inserted into a patient 102 of FIG. 4). In particular, FIG. 18 is a downward looking cutaway sectional view taken at Section A-A of FIG. 17, showing the catheter 68, two orifices 69, and an electrically conductive wire IVES electrode 70. The orifice 69 is configured as an opening having a size and shape to permit maximum fluidic penetration of urine or saline to enter the catheter 68. In one particular embodiment, the electrically conductive elements forming the electrodes 70 and sensors 90 and other sensors and electrodes 91 of FIG. 4 are comprised of electrically conducting and physiologically neutral conductors fabricated out of copper. In alternative embodiments, the electrically conductive elements may include, but are not limited to, silver, gold, platinum, stainless steel or other electrically conductive and physiologically inert metal or alloy.

In one embodiment, the electrode 70 is a single-channel electrically conductive wire. In another embodiment, the electrode 70 is formed of wires that are multi-stranded cords. Further alternative embodiments are contemplated for the electrode 70, such as multiple independent, electrically isolated conductive wires, or an electrically conductive wire mesh, without departing from the scope of the present invention.

More particularly, in one embodiment of the present invention, an IVES electrodes 70 is provided including one or more electrically conductive wires configured within one or more lumens of the catheter 68. The one or more electrically conductive wires are electrically connected to and terminate as electrically conductive wire electrodes 70 at the proximal end (i.e., facing the patient 102) of the catheter 68. The catheter 68 is configured with one or more openings or orifices 69 in the walls of the lumens, which permit passage from the outside of the catheter 68 to the inner lumens of the catheter 68. The one or more openings or orifices 69 permit maximum fluidic penetration into the one or more lumens of the catheter 68, of intrabladder urine or saline to enter the catheter 68, and contact the IVES electrodes 70. The contact between the fluid and the IVES electrodes 70 facilitates electrical contact between the IVES electrodes 70 and the intrabladder surface tissues.

As illustrated more particularly in FIG. 18, in the present embodiment, the IVES electrode 70 is fixed to the interior tip 72 of the catheter 68. The fixation of the IVES electrode 70 ensures that no errant conductive filaments extrudes from the orifice, and thereby reduces risks of electrical burns resulting by contact between the conductive filaments and intrabladder tissues.

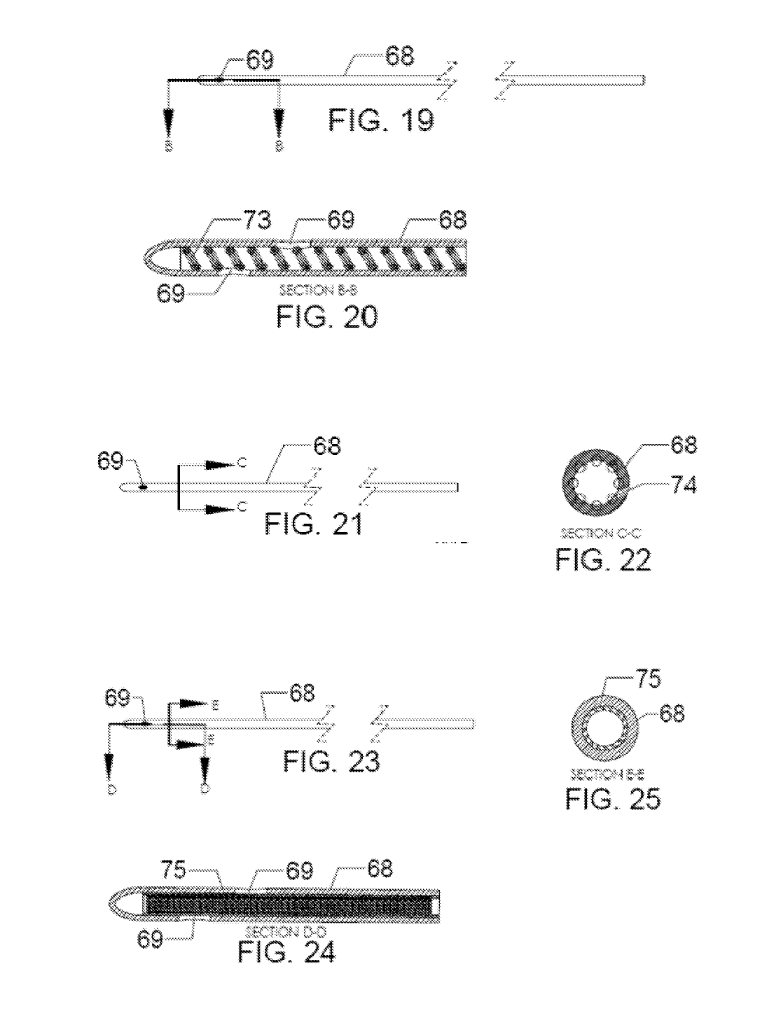

Referring now to FIGS. 19 and 20, there is shown another embodiment of catheter 68 including an orifice 69 located at the proximal end and having an IVES electrode 70. FIG. 20 is, a downward looking cutaway sectional view taken at Section B-B of FIG. 19, showing the catheter 68, two orifices 69, and an IVES electrode 73. In the present particularly illustrated embodiment, the IVES electrode 73 is an electrically conductive wire helix within, and fixated to, the interior wall of the catheter 68. The fixation of the electrically conductive wire helix IVES electrode 73 ensures that no errant conductive filaments extrudes from the orifice, and thereby reduces risks of electrical burns resulting by contact between the conductive filaments and intrabladder tissues.

FIGS. 21 and 22 show a further alternate embodiment of a catheter 68 including an orifice 69 located at the proximal end. FIG. 22 is a backward looking cutaway sectional view taken at Section C-C of FIG. 21, showing the catheter 68 and an electrically conductive wire IVES electrode 74 that is partially embedded or extruded in an inner surface wall of the catheter 68. The embedding of the electrically conductive wire IVES electrode 74 ensures that no errant conductive filaments extrudes from the orifice, and thereby reduces risks of electrical burns resulting by contact between the conductive filaments and intrabladder tissues.

FIGS. 23-25 illustrate a further alternative embodiment of a catheter 68 and an orifice 69 located at the proximal end. In particular, FIG. 24 is a downward looking cutaway sectional view taken at Section D-D, showing the catheter 68, two orifices 69, and an electrically conductive wire mesh IVES electrode 75 that is fixated to, and at least partially embedded or extruded into, the interior wall of the catheter 68. FIG. 25 is a backward looking cutaway sectional view taken at Section E-E of FIG. 23, showing the catheter 68 and the electrically conductive wire mesh IVES electrode 75 that is fixated to, and embedded within, the interior wall of the catheter 68. The fixation and embedding of the IVES electrode 75 ensures that no errant conductive filaments extrudes from the orifice, and thereby reduces risks of electrical burns resulting by contact between the conductive filaments and intrabladder tissues

Alternative Embodiments of MultiLumen IVES Electrodes

In alternative embodiments, one or more electrically conductive wires, wire helix, wire mesh, or other electrical conductive elements carry and conduct signals include, but are not limited to, independent, electrically isolated Stimulation Parameters Output Groups.

FIG. 26 shows an alternative embodiment of a catheter 76 including two orifices 77 at the proximal end of the catheter 76 and a plurality of lumens therethrough. One or more lumens within the catheter 76 include one or electrical conductors, including, but not limited to, one or more electrical conductive wires, wire helix, wire mesh, or other electrical conductive elements.

For example, FIG. 27 is a backward looking cutaway sectional view taken at Section K-K of FIG. 26, showing the catheter 76, three individual lumens (78, 80 and 82), and three IVES electrodes (79, 81 and 83). The IVES electrodes (79, 81 and 83) of the present embodiment are configured as multiple electrically conductive wire electrodes positioned and partially embedded within the interior walls of each lumen (78, 80 and 82). Although shown as wire electrodes, the invention is not meant to be limited only thereto, as other types of electrical conductive elements could be used in individual ones of the lumens, as desired.

FIG. 28 shows a further embodiment of a catheter 76 including two orifices 77 at the proximal end of a catheter 76 having a plurality of lumens. FIG. 29 is a backward looking cutaway sectional view taken at Section J-J of FIG. 28 of the catheter 76, showing an exemplary configuration of three individual lumens (78, 80 and 82), and two IVES electrodes (84 and 85). IVES electrode 84 include electrical conductors positioned within, and fixated to, the interior wall of the first lumen 78 of the catheter 76, and an IVES electrodes 85, made up of a bundle of electrical conductors positioned and fixated to the interior wall of the second lumen 80. In the present embodiment illustrated, no IVES electrode is present within the interior of the third lumen 82.

IVES Orifice Safety Features

Referring now to FIG. 30, there is shown a downward looking cross sectional view of a catheter 68 that implements a protective mechanism to further reduce the risks of electrical burns that may result by contact between IVES conductive filaments and intrabladder tissues. FIG. 31 is a backward looking cross-sectional view taken at Section T-T of the catheter 68 of FIG. 30, showing a plurality of orifices 69, a protective mesh 86, and electrical conductors 70 within the lumen of the catheter 68. The protective mesh 86 is a non-conductive mesh fabricated from physiologically inert, pliable plastic, which is configured to encircle the inner circumference of the catheter 68 and prevent a loose conductive filament from the electrical conductors 70 from protruding out from an orifice 69 and making contact with the intrabladder surface, polyps or other intrabladder tissues. Alternately, the protective mechanism can be formed as a non-conductive, protective, non-mesh sleeve, if desired.

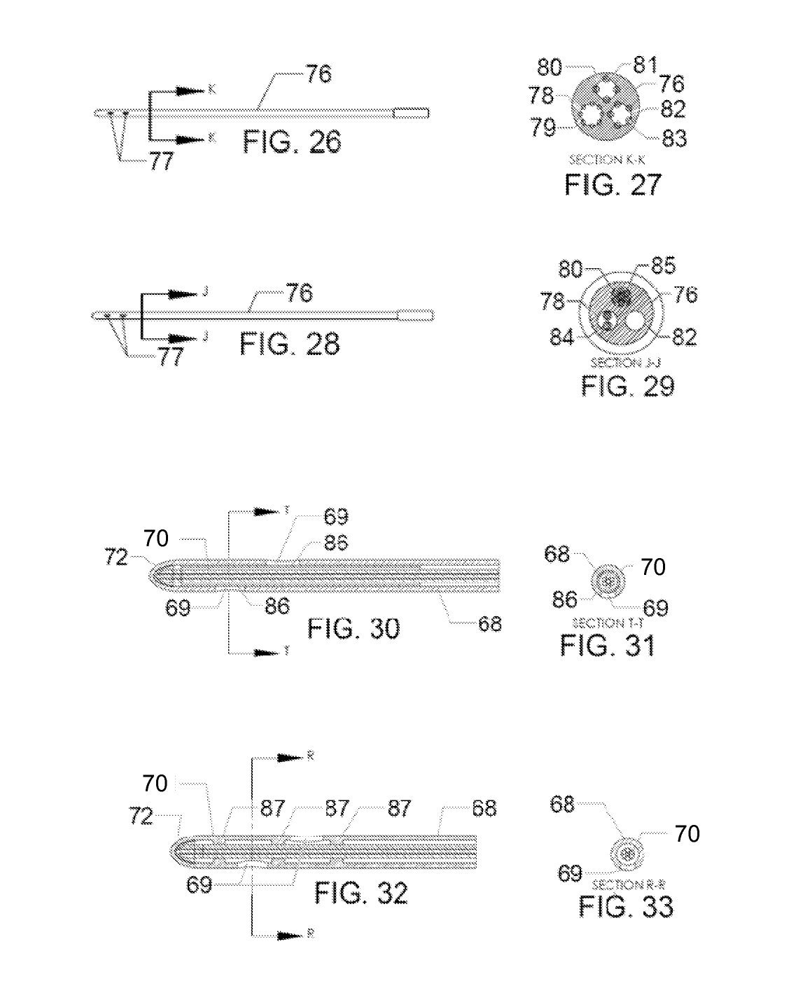

FIGS. 32 and 33 are cross sectional views of an alternative embodiment of a catheter 68 including a protective mechanism. In particular, FIG. 33 is a backward looking cross-sectional view taken at Section R-R of the catheter 68 of FIG. 32, showing the orifice 69 and the electrical conductors 70 within a lumen of the catheter 68. In the present embodiment, catheter 68 includes at least one rib 87, but more preferably, a plurality of ribs 87 positioned on the inner surface of the lumen of the catheter 68. Each of the plurality of ribs 87 has a thickness that extends from the inner surface of the catheter 68 to the outer surface of the electrical conductors 70, and a width sufficient to give structural stability to the rib 87. In one particular embodiment of the invention, the width of each rib is equal to its thickness.

As illustrated in FIGS. 32-33, the ribs 87 are positioned adjacent to, and on either side of the orifices 69. Because the ribs 87 encircle and clasp the electrical conductors 70 on either side of each orifice 69, the possibility of a loose conductive filament protruding out of an orifice 69 and making contact with the intrabladder surface, polyps or other intrabladder tissues is reduced.

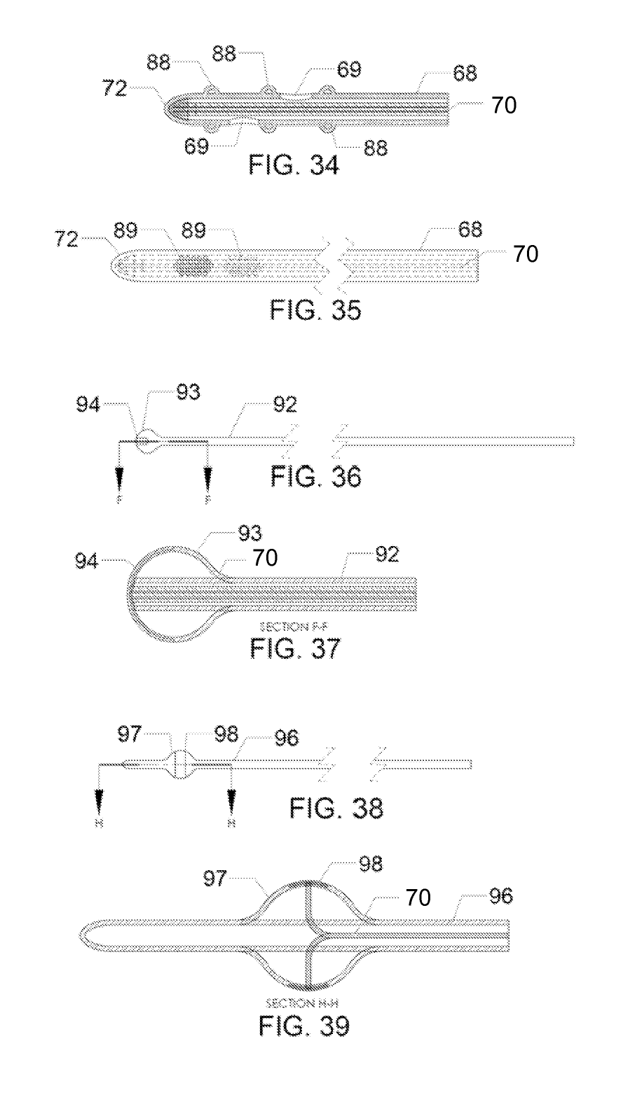

FIG. 34 is a downward looking cross sectional view of a further embodiment of a catheter 68 that implements a protective mechanism including multiple inflatable balloons 88 encircling the outer surface of the catheter 68. In one embodiment of the invention, each balloon 88 has an inner radius that extends from the outer surface of the catheter 68 to a value equal to, or about, 10% of the radius of the catheter 68. Alternately, if desired, the inflatable balloons 88 may have other configurations, such as an inner radius that extends from the outer surface of the catheter 68 to a value ranging from 10% to 50% of the radius of the catheter 68.

The inflatable balloons 88 are positioned adjacent to, and on either side of, each of the orifices 69. Because the inflatable balloons 88 encircle the catheter 68 on either side of the orifices 69, the possibility of a loose conductive filament from the electrical conductors 70 protruding out of an orifice 69 and making contact with the intrabladder surface, polyps or other intrabladder tissues is reduced.

FIG. 35 is a cross sectional view of yet another embodiment of a catheter 68 having a protective mechanism. In the present embodiment, the catheter 68 includes perforated orifice openings 89 positioned on the outer surface of the catheter 68. In one particular embodiment, each of the perforated orifice openings 89 have a diameters equal to of 2% of the diameter of a typical catheter orifice opening. However, if desired, the diameters for the perforated orifice openings 89 can range from 2% to 80% of the diameter of a typical catheter orifice opening. In one particular embodiment, the perforated orifice openings 89 can be concentrated in clusters, as illustrated, to increase the total effective size of the opening. Because the perforated orifice openings 89 are much smaller than standard orifice openings, the possibility is reduced of a loose conductive filament from electrical conductors 70 protruding out of a perforated orifice opening 89 and making contact with the intrabladder surface, polyps or other intrabladder tissues.

The IVES Sensors 90

In one particular embodiment of the invention, the IVES sensors 90 are implemented as at least one of: multiple independent, electrically isolated, electrically conductive bands; and/or single or multiple independent, electrically-isolated, electrically conductive contacts shaped in the form of a square, rectangle, circle, oval or other shape. In an embodiment, the bands or contacts are solid electrical conductors affixed to the inner or outer surface of the catheter 68.

In one particular exemplary embodiment, the sensor 90 is configured as a temperature-sensitive thermocouple, which is affixed to the inner or outer surface of the catheter 68, and which provides a temperature indication to the CPM 10. In another particular exemplary embodiment, the sensor 90 is configured as a pressure-sensitive balloon, which is affixed to the inner or outer surface of the catheter 68 to provide a pressure indicative signal to the CPM 10.

In alternative embodiments, IVES sensors 90 may be configured in the same way that IVES electrodes 70 are configured. Hence IVES sensors 90 may include, but are not limited to, one or more of an electrically conductive wire, wire helix, wire mesh, or other electrical conductive element in the same manner as IVES electrodes 70, discussed in connection with FIG. 17-FIG. 35. Such IVES sensors 90 are configured within one or more lumens of a catheter 68 and are electrically connected to, and terminate as, IVES sensors 90 within the catheter 68.

In an alternative embodiment, the catheter electrodes 70 and catheter sensors 90 are identical electrical conductors because their respective signals share the same electrical pathways. In this case, the SOM 37 and CVMM 38 multiplex channels so that only one of the SOM 37 and CVMM 38 modules, respectively, takes control of the signal path at any given time. When the ESM 8 is in drive-mode, the SOM 37 takes priority to drive its signals (i.e., the Stimulation Parameters Output Group) to the catheter electrodes 70. When the ESM 8 is in receive-mode, the CVMM 38 takes priority to receive its signals (i.e., the Measured Parameters) from the catheter sensors 90.

VES Electrodes

Referring now to FIGS. 36-37, there is provided a catheter 92 including an inflatable balloon 93 at its proximal end, and a VES electrode 94 configured as an electrically conductive band positioned laterally across the proximal end of the inflatable balloon 93. FIG. 37 is a cutaway sectional view taken at Section F-F of FIG. 36, illustrating in greater detail the catheter 92, the inflatable balloon 93, the electrically conductive band VES electrode 94. As can also be seen in FIG. 37, in the present embodiment, the electrically conductive wire electrode 70 inside the catheter 92 is fixed to, and makes electrical contact with, the interior of the electrically conductive band VES electrode 94.

In one particular embodiment, a conductive plug (such as the conductive plug 46 of FIG. 7) is additionally provided having one or more electrically conductive wires, which electrically connect with one or more corresponding electrically conductive wires within the catheter connector (43 of FIG. 7), having one or more electrodes 70. In an embodiment, a VES electrode 70 comprises one or more electrically conductive wires configured within one or more lumens of the catheter 92, which are electrically connected to and terminate as a VES electrode 70 that is electrically connected to an electrically conductive band 94 located at the proximal end (i.e., the end that contacts the patient) of a catheter 92. Thus located, the electrically conductive band 94 makes direct physical contact with the outer surface tissues of the patient in the perineum area of the pelvic floor.

Referring now to FIGS. 38 and 39, there is illustrated one particular embodiment of a catheter 96 including an inflatable balloon 97 located at the mid-section of the catheter 96, and an electrically conductive band VES electrode 98 positioned around the circumference of the inflatable balloon 97.

FIG. 39 is a cutaway sectional view taken at Section H-H of FIG. 38, showing in greater detail, the catheter 96, the inflatable balloon 97 at the mid-section of the catheter 96, and the electrically conductive band VES electrode 98 positioned around the circumference of the inflatable balloon 97. Additionally, FIG. 39 shows an electrically conductive wire electrode 70 within, which is fixed to, and makes electrical contact with, the interior of the electrically conductive band VES electrode 98. In particular the end of the electrically conductive wire electrode 70 is configured to contact the VES electrode 98, at more than one point.

VES Sensors 90

In one particular embodiment of the invention, VES sensors 90 include, but are not limited to, one or more electrically conductive wires contained within one or more lumens of the catheter 92, and electrically connected to, and terminate as, VES sensors 90 within the catheter 92, in the same manner as VES electrodes 94 or VES electrodes 98, described herein above.

In one embodiment, VES sensors 90 use standard interfaces to connect electrically to the ESM (8 of FIG. 4), and with external measurement units, including, but not limited to, vaginal electrodes and sensors that make physical contact with the perineum area of the pelvic floor and the vaginal tissues. In one particular embodiment, a vaginal electrode and sensor is formed as an electrically conductive band configured on the catheter 96 at a position to make contact with the vaginal area, in the same manner as is illustrated in FIG. 38 in connection with the VES electrode 98.

In another embodiment, VES sensors 90 are provided that use standard interfaces to electrically connect an ESM (8 of FIG. 4) with external measurement units, including, but not limited to, urethral electrodes and sensors that make physical contact with the perineum area of the pelvic floor and the urethral tissues. In an embodiment, a urethral electrode and sensor is configured as an electrically conductive band, in the same manner as illustrated in FIG. 38 in connection with the VES electrode 98, and located on the catheter 96 so as to make contact with the urethral area.

In an embodiment, VES sensors 90 use standard interfaces to connect electrically the ESM 8 with external measurement units, including anal electrodes and sensors that make contact physical contact with the perineum area of the pelvic floor and the anal tissues. In an embodiment, an anal electrode and sensor is an electrically conductive band, in the same manner as illustrated in FIG. 38 in connection with the VES electrode 98, and located on the catheter 96 so as to make contact with the anal area.

Other Sensors and Electrodes 91

In an embodiment, standard interfaces connect electrically the ESM 8 to other sensors and electrodes 91 to receive external measurement inputs, including, but not limited to, surface electrodes, implanted electrodes or special probes, which collect inputs from specific locations on the body including, but not limited to, direct and indirect measurement of electrical, mechanical and chemical activity related to the bladder and its control, such as the perineum, pelvic floor, urethral area, rectal area, a specific muscle, other areas within the urinary system, transcranial nerve region over the motor cortex or other areas, or receive EEG, EMG, ultrasonic, pressure, biological or other sensor Parameters. In an embodiment, standard interfaces connect electrically the ESM 8 to other sensors and electrodes 91 including, but not limited to, surface electrodes or implanted electrodes to deliver electrical stimulation to specific locations on the body including, but not limited to, the perineum, pelvic floor, urethral area, rectal area, a specific muscle or other areas within the urinary system.

Remote Feedback Module 100

The feedback response mechanism of the present embodiment includes, but is not limited to, the RFM 100 and the RSM 101. The RFM 100 collects responses to a questionnaire that the patient answers by running a software application on a personal device, such as a smartphone or tablet. In one particular embodiment, the personal device of the patient is configured to run a software application that provides a questionnaire to the patient via a graphical user interface (GUI) of the personal device. The RFM 100 uploads information via the internet or some other connection mechanism to the RSM 101. The RSM 101 collates input responses into an aggregated database for use by the physician to update historical clinical result tabulations and develop treatment models. The input responses include, but are not limited to, feedback responses by one or more patients, as well as stimulation and measured parameters uploaded by one or more ESM units.

Returning to FIG. 4, the figure illustrates an embodiment of the RFM 100, which comprises application software (“App”) running on a mobile computing device, personal digital assistant, smart phone or similar device, which executes a questionnaire that the patient 102 answers. The patient 102 provides inputs including, but not limited to, quality of life responses, initial diagnosis, weight, incontinence episodes, progression of other indicators such as patient Baseline Stimulation Parameters and threshold settings, and other trend indicators relative to any biological parameters. The RFM 100 transmits the patient's 102 information along with patient 102 identification (“Patient ID”), system identification (“System ID”), and a system-generated date/time stamp (“System Timestamp”) (cumulatively, “Feedback Response Parameters”). The RFM 100 transmits the Feedback Response Parameters from the RFM 100 to the RSM 101 over an internet or other wired or wireless connectivity scheme. In an embodiment, Feedback Response Parameters also include, but are not limited to: initial diagnosis; progression of responses as defined by the patient 102 or clinician 7 after each therapy via the survey questionnaire, biological-response related questions or other mutually-defined indicator; and progression of patient's 102 pain threshold after each therapy.

Remote Server Module 101

Returning to FIG. 4, the figure illustrates an embodiment of the RSM 101. In an embodiment, the RSM 101 comprises an independent, centrally located application and storage server, connected with ESMs 8 in the field and other compute devices (such as standalone computers or RFMs 100) as configured via an internet or other wired or wireless connectivity scheme. The RSM 101 initially stores lookup tables representing prior historical clinical results (in the form of Baseline Stimulation Parameter values) as a function of patient condition diagnoses and treatment outcome objectives (e.g., Urge UI/Hypertonic Detrusor, or Stress UI/Hypotonic Detrusor), updated Adjustment Functions, and other representations of mathematical performance models describing the relationships among this information (collectively, “Clinical Information”). The RSM 101 also receives Feedback Response Parameters transmitted from all RFMs 100 operating in the field. Over the course of therapies, the RSM 101 receives updated Feedback Response Parameters uploaded by all ESMs 8 and RFMs 100 operating in the field, Clinical Information updated by clinicians 7, and stores information. The RSM 101 stores information on a patient-specific basis by associating the Patient ID with the patient-specific information. The RSM 101 also stores the information on an aggregated, anonymous basis to build a cumulative historical database of all patient information (“Aggregated Parameter Models”).

In an embodiment, based on the results stored in the RSM 101, a clinician 7 downloads the Stimulation Parameter Inputs for analysis. Based on this information, the clinician 7 performs statistical regression analyses and other analyses to update the mathematical performance models, predict updated Therapy Modalities, optimize treatment algorithms, and update preprogrammed Stimulation Parameter values that correspond to these updates and optimizations.

In an embodiment, the clinician 7 uploads revised Stimulation Parameter Inputs into the RSM 101. When an ESM 8 in the field connects to the RSM 101, the ESM 8 retrieves updated information via its UIM 9 and updates its designated memory in the MM 11. The ESM 8 then executes updated treatments corresponding to the revised Stimulation Parameter Inputs (“Therapy Optimization”).

Method of Use:

Referring now to FIGS. 2A-39, the system can be used to perform a method one particular embodiment of which is described below.

1. Initial Diagnosis/Initial Therapy Settings.

In an embodiment of this invention, the clinician 7 considers the patient's 102 condition and diagnosis, determines treatment objectives, and selects a treatment modality. In OLM 15, the clinician 7 selects preconfigured electrical Stimulation Parameter Inputs corresponding to the treatment modality as specified by historical clinical result tabulations stored in the MM 11, e.g., for a particular diagnosis of hyper tonic detrusor, the historical clinical result table may specify Baseline Stimulation Parameters known to inhibit contractions, inhibit and activate detrusor contractions, and relax the sphincter orifice.

Choosing the appropriate initial Baseline Stimulation Parameters also depends on the patient's 102 feedback. For example, during the initial session, the clinician 7 administers stimulation therapy specified by the Stimulation Parameters Output Group including, but not limited to, a certain voltage, current, pulsewidth, frequency, waveform shape and waveform phase. Immediately following the initial session, the patient 102 tells the clinician 7 how he or she feels, and whether he experienced any pain or discomfort. The clinician 7 and patient 102 may run through several iterations of OLM 15 while establishing an appropriate starting voltage, current, and other settings based on the diagnosis as well as the patient's 102 verbalized thresholds for discomfort or pain.

The clinician 7 adjusts the initial Baseline Stimulation Parameters as needed, and keys in the settings (either in whole, using OLM 15, or in part, using CLM 20).

2. Subsequent/Closed-Loop Therapy Settings

Following initialization and selection of the Baseline Stimulation Parameters, the clinician 7 switches the ESM 8 to CLM 20 so that the ESM 8 can determine (based on rules established set in its programming) and automatically administer subsequent Stimulation Parameters. As discussed above, an embodiment of the ESM 8 in CLM 20 calculates subsequent Stimulation Parameter values as a function of the parameters: 1) measured and fed back as inputs to the FIM 39; 2) provided as patient-specific feedback responses collected by the RFM 100 and collated by the clinician 7 via the RSM 101; 3) obtained as patient-specific measured parameters stored in the MM 11; and 4) retrieved as historical clinical results stored in the RSM 101 and the MM 11. Based on these inputs, the CPM 10 executes appropriate algorithms in the AM 12 to determine updated Stimulation Parameters to deliver more efficacious therapy treatment settings to the patient 102 in subsequent treatment cycles.

3. Using the One-Time Use Connector 43, Catheter 68, 76, 92, 96 and Other Sensors and Electrodes 91

In one particular embodiment of the invention, consistent with the clinician's 7 diagnosis of the patient 102 and prescribed treatment modality, the clinician 7 or patient 102 inserts the catheter 68, 92, 76 or 96 into the patient 102, checks the patient's 102 bladder fluid level and injects saline or drains urine as appropriate, connects any other external sensors 91 to the appropriate locations on the patient 102 as needed for the treatment, and physically connects the ESM connector 42 to the catheter connector 43 prior to a treatment session.

4. Patient Monitoring and Use of PSS 40

In one embodiment, while the ESM 8 administers the treatment session to the patient 102, the patient 102 holds the PSS 40 switch in his hand. The patient 102 monitors his reaction to the therapy and, if he experiences any pain beyond his tolerance threshold, he may abort the treatment by pressing the PSS 40.

5. During Treatment: System Measurements

In an embodiment, during the treatment session in CLM 20, the CPM 10 determines the values for subsequent Stimulation Parameters Output Groups based on the algorithms executed in the AM 12, and drives them to the patient 102 via the SOM 37 for the duration of the therapy session.

When the ESM 8 is set to run in CLM 20, if the CPM 10 determines that the Therapy Modality should be adjusted (e.g., requiring the clinician 7 or patient 102 to place additional external, other sensors and electrodes 91 on the perineum or other surface or musculature areas of the patient 102), then the CPM 10 halts the algorithm. The UIM 9 displays an appropriate message on the display to require the clinician 7 or patient 102 to take appropriate action. The ESM 8 waits until the clinician 7 or patient 102 signals the CPM 10 to resume operation by keying in the appropriate command into the UIM 9, then the CPM 10 continues running in CLM 20.

6. Post-Treatment: Disconnecting the Catheter 68, 76, 92, 96

In an embodiment, following the treatment session, the clinician 7 or patient 102 withdraws the catheter 68, 76, 92, 96 from the patient 102 and disconnects the catheter connector 43 from the ESM connector 42, which severs the electrical and physical connections. As described above, after disconnection, the catheter connector 43 locks itself and blocks any subsequent attempts to re-insert and connect the ESM connector 42 with the catheter connector 43 and prevents any re-use of the catheter 68. The clinician 7 or patient 102 disposes of the catheter 68, 76, 92, 96 into a hazardous waste disposal receptacle.

7. Post-Treatment: Patient Feedback Responses

In an embodiment, following a treatment session, the patient 102 provides Feedback Response Parameters based on his perception of his condition. The patient 102 provides the feedback information by answering a survey questionnaire by running a particularly tailored software application (“App”) on his personal device, such as a smartphone, tablet, etc. Patient data (interpretive responses) include, but are not limited to:
The initial diagnosis;
Progression as defined by patient 102 on the App or by a clinician 7 after each therapy using multiple key indicators;
Patient thresholds relative to prior sessions; and
Patient response trends relative to any biological parameter.

The RFM 100 uploads the information to the RSM 101.

8. Post-Treatment: Optimizing Treatment Values and Algorithms

In one particular embodiment, following a treatment session, the clinician 7 uses information collected by the patient 102 and other information aggregated by the RSM 101 to analyze and update clinical result tabulations and to develop revised treatment models. If desired, a clinician 7 may download the results stored in the RSM 101, for analysis. Based on this information, the clinician 7 performs statistical regression analyses and other data analyses to update the Clinical Information stored in the RSM 101.

9. Uploading Updated Treatment Values and Algorithms to the RSM 101 for Downloading and Use by ESM 8 in the Field

In one particular embodiment, after the clinician 7 uploads revised Clinical Information to the RSM 101, ESMs 8 in the field later access the revised Clinical Information. The ESMs 8 establish a data connection to the RSM 101 via their UIMs 9, download the revised Clinical Information, and store it into designated MM 11 memory locations for use by the CPM 10 and its algorithms in subsequent stimulation treatment therapy.

The embodiments illustrated herein are described in sufficient detail to enable those skilled in the art to practice the teachings disclosed. Elements of any embodiment described herein can be used with, or in place of, elements of any other embodiment described herein. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by any appended claims, along with the full range of equivalents to which such claims are entitled.

The present invention provides an electrical neuromodulation stimulation system and method for treating urinary incontinence, as described herein. Accordingly, while a preferred embodiment of the present invention is shown and described herein, it will be understood that the invention may be embodied otherwise than as herein specifically illustrated or described, and that within the embodiments certain changes in the detail and construction, as well as the arrangement of the parts, may be made without departing from the principles of the present invention as defined by the appended claims.

1. An electrical neuromodulation stimulation system for treating urinary incontinence of a patient, comprising:
an electrical stimulation module configured to determine electrical stimulation therapy modalities as a function of inputs and generate a stimulation output;
a catheter including at least one stimulation electrode, said catheter connected to said electrical stimulation module to provide an electrical stimulation treatment to the patient in accordance with the stimulation output generated by the electrical stimulation module;
a feedback mechanism configured to provide the electrical stimulation module with feedback related to an electrical stimulation treatment provided to the patient; and
said electrical stimulation module configured to modify the determined electrical stimulation therapy modality for a subsequent treatment of the patient based on the feedback received via the feedback mechanism.

2. The system of claim 1, wherein the feedback mechanism includes at least one sensor contained in said catheter.

3. The system of claim 1, wherein the feedback mechanism includes at least one sensor external to said catheter.

4. The system of claim 1, wherein the feedback mechanism includes a remote device into which the patient manually enters feedback regarding biological information.

5. The system of claim 4, wherein the remote device is a personal device of the patient executing a software application that provides a questionnaire to the patient via a graphical user interface of the personal device; said questionnaire relating to a prior electrical stimulation treatment performed on the patient.

6. The system of claim 5, wherein information of the responses to the questionnaire, or generated from the responses to the questionnaire are provided to the electrical stimulation module via a remote server module.

7. The system of claim 5, wherein the electrical stimulation module optimizes stimulation treatment parameters used to generate a stimulation output based on patient specific measured parameters in combination with information obtained from patient specific responses to the questionnaire.

8. The system of claim 1, wherein the electrical stimulation therapy modality includes an intravesical electrostimulation (IVES) therapy modality.

9. The system of claim 8, wherein the electrical stimulation therapy modality also includes at least one type of therapy modality other than an IVES therapy modality.

10. The system of claim 1, wherein the at least one stimulation electrode of said catheter is at least one of a single-channel electrically conductive wire, a multi-stranded cord, multiple independent, electrically isolated conductive wires, or an electrically conductive wire mesh.

11. The system of claim 10, wherein the at least a portion of the one stimulation electrode is spaced from the inner wall of the catheter by a protective mechanism.

12. An electrical neuromodulation stimulation method for treating urinary incontinence of a patient, comprising the steps of:
providing the system of claim 1;
entering Baseline Stimulation Parameters into a user interface module of the electrical stimulation module;
inserting the catheter into a desired location in the patient;
generating stimulation outputs based on the Baseline Stimulation Parameters;
conveying stimulation outputs to the patient as part of a treatment;
receiving feedback at the electrical stimulation module related to the treatment provided to the patient;
subsequently, calculating next-state values for the stimulation parameters based on the received feedback; and
generating stimulation outputs for the patient using the next-state values of the stimulation parameters.

13. The method of claim 12, wherein the feedback includes at least one of patient specific measured parameters obtained during the treatment and information obtained from patient specific responses to a questionnaire.

14. The method of claim 12, wherein the feedback includes patient specific measured parameters obtained during the treatment and information obtained from patient specific responses to a questionnaire.

15. A catheter for electrical neuromodulation stimulation, comprising:
a catheter including a conductor, a body and a tip, said catheter including at least one orifice proximal to the tip, said orifice having a size and shape to permit fluidic penetration of urine or saline to enter the catheter;
the catheter additionally including a catheter connector configured to receive a connector from an electrical stimulation module; and make an electrical contact with the conductor;
the catheter connector configured to permit the insertion of the electrical stimulation module connector; and
said catheter connector further including a barrier lock mechanism configured to block the insertion of any other electrical stimulation module connector once a first electrical stimulation module connector has been full inserted into the catheter connector and subsequently retracted from the catheter connector.

16. The catheter of claim 15, wherein the barrier lock mechanism includes a lock pin that slides horizontally across a vertical axial direction of a housing channel lumen to block any further subsequent insertion of an electrical stimulation module connector into the catheter connector, after removal of a first electrical stimulation module connector.

17. The catheter of claim 16, wherein,
the barrier lock mechanism additionally includes a barrier pin lock axially aligned and concentrically fit within the housing channel lumen prior to insertion of the first electrical stimulation module connector, and a barrier pin fit concentrically within a hollow axis of the barrier pin lock;
prior to insertion of the first electrical stimulation module connection, said barrier pin prevents said lock pin from sliding across the housing channel lumen;
insertion of said first electrical stimulation module connector pushes said barrier pin into said barrier pin lock; and
removal of said first electrical stimulation module connector with said barrier pin pushed into said barrier pin lock permits said lock pin to slide horizontally across the housing channel lumen, blocking any further subsequent insertion of an electrical stimulation module connector into the catheter connector.

18. A catheter for performing an electrical stimulation treatment, comprising:
the catheter including a lumen and at least one orifice proximal to a tip of the catheter;
at least one electrically conductive wire electrode partially embedded or extruded in at least a portion of an inner surface wall of the catheter.

19. The catheter of claim 18, wherein the at least one electrically conductive wire electrode is a wire mesh.

20. The catheter of claim 18, wherein the catheter includes an inflatable balloon and the at least one electrically conductive wire electrode terminates as a VES electrode that is electrically connected to an electrically conductive band located at a proximal end of the catheter.

21. An electrical neuromodulation stimulation system for treating urinary incontinence of a patient, comprising:
an electrical stimulation module configured to determine electrical stimulation therapy modalities as a function of inputs and generate a stimulation output;
a catheter including at least one stimulation electrode, said catheter connected to said electrical stimulation module to provide an electrical stimulation treatment to the patient in accordance with the stimulation output generated by the electrical stimulation module; and
wherein said electrical stimulation therapy modalities determined by said electrical stimulation module include an IVES therapy modality combined with at least one of a paired associative stimulation (PAS) therapy modality or an electrical stimulation therapy modality.

22. The system of claim 21, wherein the electrical stimulation therapy modalities include an IVES therapy modality combined with a PAS therapy modality.

23. The system of claim 21, wherein the electrical stimulation therapy modalities include an IVES therapy modality combined with microstimulation.

24. An electrical neuromodulation stimulation method for treating urinary incontinence of a patient, comprising the steps of:
providing the system of claim 21;
conveying a stimulation treatment to the patient, the stimulation treatment including an IVES therapy modality combined with at least one of a PAS therapy modality or a microstimulation therapy modality.
